(19) United States
(12) Patent Application Publication
(10) Pub. No.: US 2026/0013457 A1
(43) Pub. Date: Jan. 15, 2026

(54) HERBICIDE-TOLERANT, SEASHORE PASPALUM 'UGA 17-726' AND PROGENY

(71) Applicant: University of Georgia Research Foundation, Inc., Athens, GA (US)

(72) Inventor: Paul RAYMER, Milner, GA (US)

(21) Appl. No.: 19/268,208

(22) Filed: Jul. 14, 2025

Related U.S. Application Data
(60) Provisional application No. 63/670,563, filed on Jul. 12, 2024.

(51) Int. Cl. A01H 6/46; A01H 5/10

(52) U.S. Cl. CPC A01H 6/4648; A01H 5/10

(57) ABSTRACT

Herein provided is a new seashore paspalum variety designated ‘UGA 17-726’ as well as the seeds, plants, and derivatives of the new seashore paspalum variety ‘UGA 17-726’. Methods for producing seashore paspalum seed by crossing the new paspalum variety with other seashore paspalum varieties and plants produced by such methods are provided. ‘UGA 17-726’ is a unique seashore paspalum having a combination of high numbers of flowering spikes, turf quality traits, high levels of salt tolerance and a mutation conferring resistance to several ACCase-inhibiting herbicides.

### CROSS-REFERENCE TO RELATED APPLICATION[S]

This application claims priority to, and the benefit of, U.S. Provisional Application No. 63/670,563, filed on Jul. 12, 2024, the entire contents of which are incorporated by reference as if fully set forth herein.

### FIELD

This disclosure provides a new and distinctive seashore paspalum variety, ‘UGA 17-726’, which is a turfgrass resistant to ACCase herbicides used for control of bermudagrass and other weedy grasses.

### BACKGROUND

Turfgrass plays a pivotal role in creating well-manicured landscapes while concurrently promoting ecological sustainability. It aids in erosion prevention, improves soil health, and carbon sequestration. However, challenges arise when undesirable and troublesome weeds invade these carefully cultivated grasses and they are subjected to excess salinity and harsh climates, compromising their purity and leading to potential contamination. Through extensive research in the field, DNA fingerprinting has been found to offer a solution by ensuring the authenticity of turfgrass varieties. Given the visual similarities among many turfgrass types, DNA fingerprinting enables accurate differentiation and assessment of these varieties.

Seashore paspalum is a warm-season turfgrass that is adapted to coastal environments. This species is generally adapted to the same regions of the world as bermudagrass [Cynodon dactylon (L.) Pers.] and has numerous morphological characteristics that make it desirable as a turfgrass. Interest in the use of seashore paspalum as a turfgrass is largely related to its tolerance to salt and other abiotic stresses. Golf course architects recommend seashore paspalum for new courses in tropical or subtropical coastal areas where salt or water quality are issues. Many existing golf courses around the world have replaced bermudagrass with paspalum.

The main difficulty in replacing bermudagrass with paspalum is bermudagrass re-establishment. Bermudagrass is highly competitive and difficult to eradicate once established. Invasion by bermudagrass and other weedy grasses can greatly reduce the aesthetic value and quality of paspalum turf. Currently there are no herbicides available that selectively control bermudagrass in seashore paspalum. Development of herbicide-resistant paspalum could provide an effective means of managing bermudagrass and other weedy grasses in paspalum and allow golf courses and sporting venues to transition from bermudagrass to seashore paspalum.

Sethoxydim is a graminicide used to control perennial and annual grasses in numerous agricultural and ornamental crops. This herbicide is an acetyl coenzyme A carboxylase (ACCase) inhibitor and a member of the cyclohexanedione (CHD) family. Sethoxydim has been marketed in the United States under numerous trade names, including Poast, Poast Plus, Segment, and Vantage (BASF Corp., Research Triangle Park, NC). Sethoxydim and other ACCase inhibitors cause cell death by competing with acetyl-CoA for its binding site in ACCase. Therefore, the first step of fatty acid biosynthesis is halted and the product, malonyl coenzyme A, is not produced. Cell membranes are compromised without fatty acids and cell death ensues.

Spontaneous occurrence of sethoxydim resistance has been observed in numerous weedy species. Such resistance has arisen after the large-scale application of sethoxydim on an annual or semi-annual basis in various cropping systems. The most reported mutation is a single Ile to Leu substitution at amino acid position 1781 in the carboxyl transferase (CT) domain of the ACCase gene. An adenine to thymine transversion in the first position of the codon accounts for this substitution. Additionally, a mutation at the 2078 codon, causing a change from aspartate (Asp) to glycine (Gly), also confers resistance to sethoxydim. Both mutations also confer resistance to aryloxyphenoxypropionate (APP) herbicides, another group of ACCase inhibiting herbicides.

Transgenic approaches for obtaining sethoxydim resistance are not practical. First, the ACCase gene is very large with an estimated size of =12,000 bp, which makes it difficult to manipulate with current transformation procedures. The cDNA sequences available from GenBank are also large (=7,000 bp) not including the vector backbone. Secondly, there is a high level of homology that exists between the inserted gene and the native genes. Since only 20 to 26 bp of homology are needed to cause gene silencing of both the inserted gene and the native gene, this approach would likely result in silencing and be lethal.

The ACCase exists in two forms: eukaryotic and prokaryotic. The eukaryotic form is a single polypeptide with distinct functional domains and is encoded by a nuclear gene. Sasaki et al. found that ACCase is compartmentalized in most plants, such that the prokaryotic form is in the chloroplast and the eukaryotic form in the cytosol. The Poaceae are unique in that they have the eukaryotic form of ACCase in both the cytosol and chloroplast. The two eukaryotic forms of ACCase in grasses are very similar, as are the genes that code for them. Even though there is homology between the plastidic and cystolic ACCases, the cystolic form is not affected by ACCase-inhibiting herbicides. The prokaryotic form is made up of four subunits and is coded by four genes, one of which is in the chloroplast genome. Although the prokaryotic ACCase is inherently herbicide resistant, engineering grasses with these genes is not a practical option, as it could require the coordinated transformation of up to four genes into the nuclear and chloroplastic genomes.

Seashore paspalum is particularly susceptible to invasion by bermudagrass and other weedy grasses in locations such as golf courses. Invasion by these grasses results in reduced aesthetic value of the turfgrass and can also impact playing quality of the courses. Seashore paspalum varieties resistant to ACCase-inhibiting herbicides used to control such invasive grasses are needed. Ideally, the seashore paspalum varieties would also have high levels of salt tolerance, high numbers of flowering spikes, and turf quality traits.

### SUMMARY

Herein is provided a new seashore paspalum variety designated ‘UGA 17-726’ as well as the seeds, plants, and derivatives of the new seashore paspalum variety ‘UGA 17-726’. Methods for producing seashore paspalum seed by crossing the new paspalum variety with other seashore paspalum varieties and plants produced by such methods are provided. ‘UGA 17-726’ is a unique seashore paspalum having a combination of high numbers of flowering spikes, turf quality traits, high levels of salt tolerance and a mutation conferring resistance to several ACCase-inhibiting herbicides.

The foregoing and other objects and features of the disclosure will become more apparent from the following detailed description.

### BRIEF DESCRIPTION OF THE DRAWINGS

Further aspects of the present disclosure will be more readily appreciated upon review of the detailed description of its various embodiments, described below, when taken in conjunction with the accompanying drawings.

FIG. 1 shows banding patterns of DNA extracted from Mauna Kea (parent line) and 23 lines derived from tissue culture following selection for sethoxydim resistance. Three SSR primers, Pv3, Pv35, and Pv51, known to be polymorphic on Paspalum vaginatum Swartz were used to illustrate distinct differences in DNA from the original parental line in nine of 23 tissue culture derived lines. Arrows indicate genotypes that differ from the parental line.

FIG. 2 shows response rate to sethoxydim in increasing amounts for the parent strain of UGA 17-726, a tissue culture control, and cultivar SR 11.

FIG. 3 shows plant injury 14 days after treatment with various amounts of sethoxydim for the plant cultivars in FIG. 2.

FIGS. 4A-4C show field experiments conducted using a control (no herbicide), clethodim at 1× and 3× (280 g/ha and 831 g/ha, respectively), fenoxyprop at 1× and 3× (191 g/ha and 372 g/ha, respectively), and sethoxydim at 1× and 3× (314 g/ha and 942 g/ha, respectively).

FIG. 5 is a photograph showing herbicide resistant seashore paspalum survives application of herbicide while invading bermudagrass does not.

FIG. 6 shows response of seashore paspalum to ACCase inhibiting herbicides after 28 days.

FIG. 7 shows response of seashore paspalum to ACCase inhibiting herbicides after 28 days.

FIG. 8 shows dose response of seashore paspalum to pinoxaden after 14 days.

FIG. 9 shows results of an herbicide trial on seashore paspalum versus bermudagrass.

FIG. 10 shows herbicide injury to 2-week-old seashore paspalum seedlings.

FIG. 11 shows response of 2-week-old seashore paspalum seedlings to injury with fusillade II after 14 days.

FIG. 12 shows response of 2-week-old seashore paspalum seedlings to injury with fusillade II after 28 days.

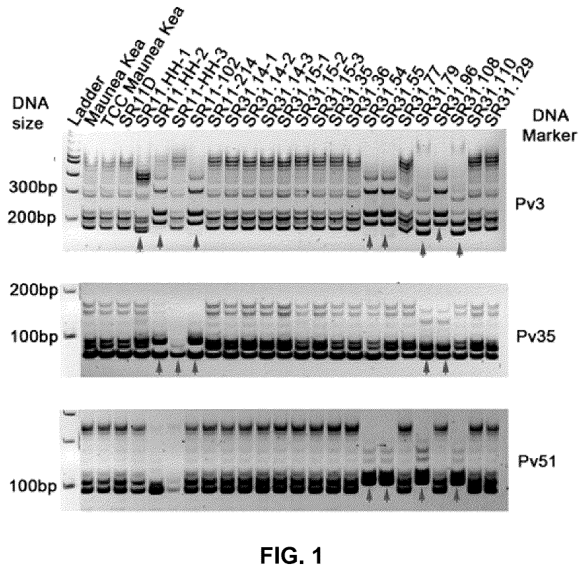

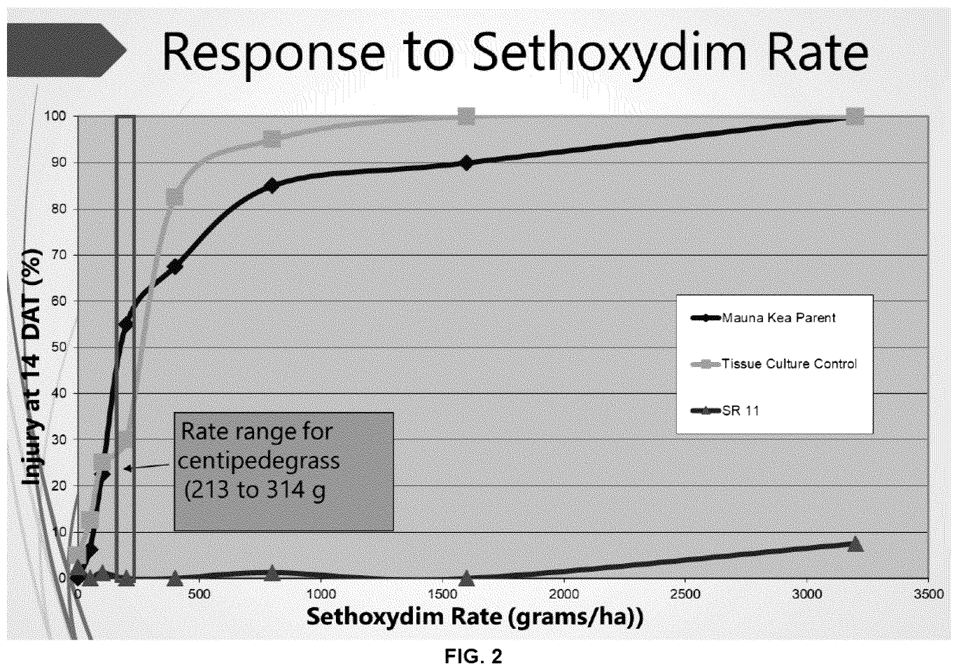

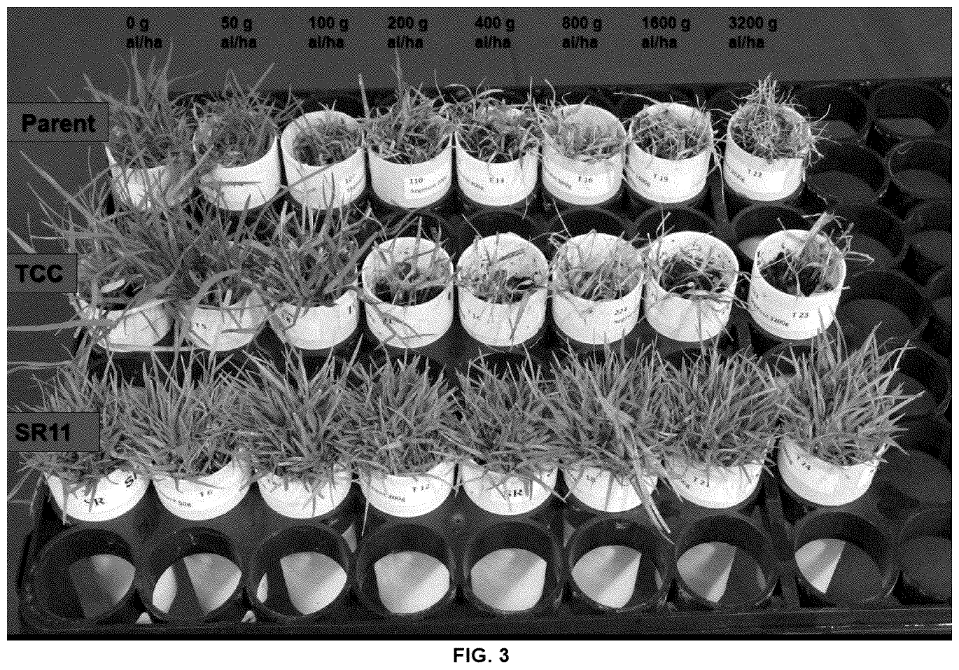

### DETAILED DESCRIPTION

The appearance of resistant alleles in response to herbicide applications indicates that resistance can be obtained without transformation. Sethoxydim resistance was obtained in maize (Zea mays L.) by in vitro selection, providing an example of a graminaceous crop in which weedy grasses can be controlled via herbicide resistance. The resistance is now understood to originate from the single Ile to Leu substitution at position 1781 mutation occurring in the plastidic ACCase in grasses.

Research initiated at the University of Georgia to induce and identify sethoxydim-resistant mutants in seashore paspalum suitable for use in a breeding program for the development of herbicide-resistant cultivars. Callus was induced from immature inflorescences then plated on callus induction medium containing 10 UM sethoxydim for selection. In vitro selection and regeneration protocols were used to select for naturally occurring mutations conferring herbicide resistance and were successful in developing a novel source of resistance to sethoxydim in seashore paspalum (Paspalum vaginatum Swartz). Green plants were regenerated from resistant callus, the Ile to Leu mutation documented, and expression of herbicide resistance confirmed. After screening more than 20,250 calli, SR cells were identified and ultimately regenerated seashore paspalum plants highly resistant to sethoxydim. The mutation conferring herbicide resistance was characterized as a single nucleotide change in the codon for position 1781 of ACCase. This mutation was estimated to occur once in each 1.74 billion cells but only 2 of the 5.22 billion cells screened resulted in regenerable plants with the mutation. Plants regenerated from these two cell lines were designated as SR 11 and SR 31.

More than two thousand individual plants were generated from these cell lines. These plants were initially evaluated for their breeding potential. Many of the plants regenerated were chimeric and genetically unstable due to large numbers of additional deleterious mutations associated with their tissue culture origin. Most plants had very poor vigor and lacked fitness. Furthermore, all regenerated plants were sterile and either failed to flower or if they did flower, failed to produce viable seed when crossed with other seashore paspalum varieties.

In one aspect, the UGA 17-726 cultivars are resistant to sethoxydim, fenoxaprop, fluazifop-butyl, pinoxaden, and other ACCase-inhibiting herbicides. In another aspect, use of these herbicides on the disclosed UGA 17-726 cultivars can be effective in controlling annual and perennial weedy grasses including, but not limited to, bermudagrass, crabgrass, goosegrass, tropical signal grass, and others.

### Description of Terms

The following explanations of terms and methods are provided to better describe the present disclosure and to guide those of ordinary skill in the art in the practice of the present disclosure. As used herein, “comprising” means “including” and the singular forms “a” or “an” or “the” include plural references unless the context clearly dictates otherwise. For example, reference to “comprising a plant” includes one or a plurality of such plants. The term “or” refers to a single element of stated alternative elements or a combination of two or more elements unless the context clearly indicates otherwise. For example, the phrase “A or B” refers to A, B, or a combination of both A and B. Furthermore, the various elements, features and steps discussed herein, as well as other known equivalents for each such element, feature or step, can be mixed and matched by one of ordinary skill in this art to perform methods in accordance with principles described herein. Among the various elements, features, and steps some will be specifically included and others specifically excluded in particular examples.

Unless explained otherwise, all technical and scientific terms used herein have the same meaning as commonly understood to one of ordinary skill in the art to which this disclosure belongs. Although methods and materials similar or equivalent to those described herein can be used in the practice or testing of the present disclosure, suitable methods and materials are described below. The materials, methods, and examples are illustrative only and not intended to be limiting. All references cited herein are incorporated by reference in their entireties.

In some examples, the numbers expressing quantities of ingredients, properties such as molecular weight, reaction conditions, and so forth, used to describe and claim certain embodiments are to be understood as being modified in some instances by the term “about” or “approximately.” For example, “about” or “approximately” can indicate +/−20% variation of the value it describes. Accordingly, in some embodiments, the numerical parameters set forth herein are approximations that can vary depending upon the desired properties sought to be obtained by a particular embodiment. Notwithstanding that the numerical ranges and parameters setting forth the broad scope of some examples are approximations, the numerical values set forth in the specific examples are reported as precisely as practicable. The recitation of ranges of values herein is merely intended to serve as a shorthand method of referring individually to each separate value falling within the range.

Backcross: The mating of a hybrid to one of its parents. For example, hybrid progeny, for example a first-generation hybrid (F₁), can be crossed back one or more times to one of its parents. Backcrossing can be used to introduce one or more single locus conversions (such as one or more desirable traits) from one genetic background into another.

Cell: Cell as used herein includes a plant cell, whether isolated, in tissue culture or incorporated in a plant or plant part.

Cotyledon: A type of seed leaf. The cotyledon contains the food storage tissues of the seed.

Cross: Synonymous with hybridize or crossbreed. Includes the mating of genetically different individual plants, such as the mating of two parent plants.

Cross-pollination: Fertilization by the union of two gametes from different plants.

F₁ hybrid: The first generation progeny of the cross of two nonisogenic plants.

Flower: Refers to all parts of the flower, including but not limited to, stigma, style, ovary, anther, filament, corolla, and calyx.

Gene: Refers to a segment of nucleic acid. As used herein a “gene” can refer to a hereditary unit corresponding to a sequence of DNA that occupies a specific location on a chromosome and that contains the genetic instruction for a characteristic(s) or trait(s) in an organism. The term gene can refer to translated and/or untranslated regions of a genome. “Gene” can refer to the specific sequence of DNA that is transcribed into an RNA transcript that can be translated into a polypeptide or be a catalytic RNA molecule, including but not limited to, tRNA, siRNA, piRNA, miRNA, long-non-coding RNA and shRNA. A gene can be introduced into a genome of a species, whether from a different species or from the same species, using transformation or various breeding techniques. In some examples, a gene encodes a desirable trait, such as disease, insect, or herbicide resistance.

Gene Silencing: A general term describing epigenetic processes of gene regulation, including any technique or mechanism in which the expression of a gene is prevented.

Genotype: The genetic constitution of a cell, an organism, or an individual (i.e., the specific allele makeup of the individual) usually with reference to a specific character under consideration.

Maturity date: The evaluation of plants considered as mature when the highest percentage of the pods have reached the mature colors, black, brown, and orange.

Plant: Includes reference to an immature or mature whole plant, including a plant from which seed, roots or leaves have been removed. Seed or embryo that will produce the plant is also considered to be the plant.

Plant height: Plant height is taken from the top of the soil to the tip of the plant main stem, and is typically measured in centimeters or inches.

Plant parts: Includes protoplasts, leaves, stems, roots, root tips, anthers, pistils, seed, embryo, pollen, ovules, cotyledon, hypocotyl, flower, shoot, tissue, petiole, cells, pods, meristematic cells and the like. Includes plant cells of a tissue culture from which seashore paspalum plants can be regenerated.

Progeny: Offspring; descendants. Includes an F₁ seashore paspalum plant produced from the cross of two seashore paspalum plants where at least one plant includes seashore paspalum cultivar ‘UGA 17-726’, and progeny further includes, but is not limited to, subsequent F₂, F₃ F₄, F₅, F₆, F₇, F₈, F₉, and F₁₀ generational crosses with the recurrent parental line.

Regeneration: The development of a plant from tissue culture. The cells may or may not have been genetically modified. Plant tissue culture relies on the fact that all plant cells have the ability to generate a whole plant (totipotency). Single cells (protoplasts), pieces of leaves, or roots can often be used to generate a new plant on culture media given the required nutrients and plant hormones.

Relative maturity: Refers to the maturity grouping designated by the turfgrass industry over a given growing area. This figure is generally divided into tenths of a relative maturity group. Within narrow comparisons, the difference of a tenth of a relative maturity group equates very roughly to a day difference in maturity at harvest.

Resistance: The ability of a plant to prevent and/or have lower incidence of damage from herbicides, such as, but not limited to sethoxydim, fenoxaprop, fluazifop-butyl, and pinoxaden.

Seed: The part of a flowering plant that typically contains the embryo with its protective coat and stored food and that can develop into a new plant under the proper conditions. Seed can refer to a fertilized and mature ovule.

Self-pollination: The transfer of pollen from the anther to the stigma of the same plant. Single locus converted (conversion) plant: Plants developed by backcrossing and/or by genetic transformation, where essentially all of the desired morphological and physiological characteristics of a seashore paspalum variety are recovered in addition to the characteristics of the single locus transferred into the variety via the backcrossing technique.

Tissue culture: A composition that includes isolated cells of the same or a different type or a collection of such cells organized into parts of a plant.

Transformation: The introduction of new genetic material (e.g., exogenous transgenes) into plant cells. Exemplary mechanisms that are to transfer DNA into plant cells include (but not limited to) electroporation, microprojectile bombardment, Agrobacterium-mediated transformation and direct DNA uptake by protoplasts.

Transgene: A gene or genetic material that has been transferred into the genome of a plant, for example by genetic engineering methods. Exemplary transgenes include a cDNA (complementary DNA) segment, which is a copy of mRNA (messenger RNA), and the gene itself residing in its original region of genomic DNA. For instance, a transgene can refer to a segment of DNA containing a gene sequence that is introduced into the genome of a seashore paspalum plant or plant cell. This non-native segment of DNA may retain the ability to produce RNA or protein in the transgenic plant, or it may alter the normal function of the transgenic plant's genetic code. In general, the transferred nucleic acid is incorporated into the plant's germ line. Transgene can also describe any DNA sequence, regardless of whether it contains a gene coding sequence or it has been artificially constructed, which has been introduced into a plant or vector construct in which it was previously not found.

### DISCUSSION

### New Seashore Paspalum Resistant to ACCase Inhibiting Herbicides

The present disclosure relates to a new seashore paspalum variety, ‘UGA 17-726’. This new variety is ACCase inhibitor resistant and has a high yield. In some examples, the new variety is adapted to growth in areas of the United States that commonly experience high salt concentrations in soils.

In some examples, a ‘UGA 17-726’ plant or progeny thereof has a lower ACCase inhibiting herbicide susceptibility than other similar cultivars.

The disclosed ‘UGA 17-726’ plants and seeds can be used to produce other seashore paspalum plants and seeds, for example as part of a breeding program. Choice of breeding or selection methods using to generate new seashore paspalum plants and seeds can depend on the mode of plant reproduction, the heritability of the trait(s) being improved, and the type of variety used commercially (e.g., F₁ hybrid variety, pure line variety, etc.). For highly heritable traits, a choice of superior individual plants evaluated at a single location can be effective, whereas for traits with low heritability, selection can be based on mean values obtained from replicated evaluations of families of related plants. Popular selection methods commonly include pedigree selection, modified pedigree selection, mass selection, recurrent selection and backcrossing.

The complexity of inheritance influences choice of the breeding method. Backcross breeding can be used to transfer one or a few favorable genes for a highly heritable trait into a desirable variety. This approach has been used for breeding disease-resistant varieties (e.g., see Bowers et al., 1992. Crop Sci. 32(1):67-72; Nickell and Bernard, 1992. Crop Sci. 32(3):835). Various recurrent selection techniques can be used to improve quantitatively inherited traits controlled by numerous genes.

Promising advanced breeding lines can be thoroughly tested and compared to appropriate standards in environments representative of the commercial target area(s) for generally three or more years. The best or most preferred lines are candidates for new commercial varieties. Those still deficient in a few traits may be used as parents to produce new populations for further selection.

The disclosure provides seashore paspalum plants having or consisting of the morphological and physiological characteristics of ‘UGA 17-726’, such as the characteristics noted in the Examples, for example ACCase-inhibiting herbicide resistance, high salt tolerance, and the like.

Also provided are seeds of such plants, progeny of such plants, and parts of such plants (such as pollen, ovules and cells). In one example, the disclosure provides seashore paspalum plants having the genotype of ‘UGA 17-726’. For example, the disclosure provides plants produced by growing the seed of the new seashore paspalum variety ‘UGA 17-726’.

The disclosure describes a tissue culture of regenerable cells of the new seashore paspalum variety ‘UGA 17-726’ from the seeds of ‘UGA 17-726’ as described above, as well as plants regenerated therefrom. Such regenerated seashore paspalum plants can include or consist of the physiological and morphological characteristics of a plant grown from the seed of the new seashore paspalum variety ‘UGA 17-726’. Exemplary regenerable cells include but are not limited to those from protoplasts or cells, such as those from embryos, meristematic cells, pollen, leaves, roots, root tips, anther, pistil, flower, seed, cotyledon, hypocotyl, shoot, pedicel, petiole, or stem of the new seashore paspalum variety ‘UGA 17-726’.

Methods of producing seashore paspalum seed from the ‘UGA 17-726’ plants are provided. In some examples such methods include crossing ‘UGA 17-726’ with itself or a second seashore paspalum plant and harvesting a resulting turfgrass seed. In some examples, the second seashore paspalum plant has one or more desirable traits, which is/are introduced into (e.g., via transformation) plants and seeds resulting from such a cross. For example, the second plant can be transgenic, wherein the transgene confers the desirable trait(s). Seeds produced by such methods, including F₁ hybrid seeds, as well as plants or parts thereof produced by growing such a seed, are provided. In some examples, the method of crossing includes planting seeds of the new seashore paspalum variety ‘UGA 17-726’, cultivating plants resulting from the seeds until the plants bear flowers, allowing fertilization of the flowers of the plants; and harvesting seeds produced from the plants.

Methods are provided for producing a plant of seashore paspalum variety ‘UGA 17-726’ that has one or more added desired traits, as well as plants and seeds generated from such methods. In one example, such a method provides a seashore paspalum plant having a single locus conversion of the new seashore paspalum variety ‘UGA 17-726,’ wherein the plant includes or expresses the physiological and morphological characteristics of the new turfgrass variety ‘UGA 17-726.’ In some embodiments, the single locus conversion can include a dominant or recessive allele. Such methods can include introducing a transgene that confers one or more desired traits into a plant of the new seashore paspalum variety ‘UGA 17-726’ (e.g., via transformation). Exemplary desired traits include herbicide tolerance, resistance to an insect, resistance to a bacterial disease, resistance to a viral disease, resistance to a fungal disease, resistance to a nematode, resistance to a pest, male sterility, site-specific recombination; abiotic stress tolerance (such as tolerance to drought, heat, cold, low or high soil pH level, and/or salt); or other improved nutritional qualities.

Methods of introducing a single locus conversion (such as a desired trait) into the new seashore paspalum variety ‘UGA 17-726’ are provided. In some examples the methods include (a) crossing a plant of variety ‘UGA 17-726’ with a second plant having one or more desired traits to produce F₁ progeny plants; (b) selecting F₁ progeny plants that have the desired trait to produce selected progeny plants; (c) crossing the selected progeny plants with at least a first plant of variety ‘UGA 17-726’ to produce backcross progeny plants; (d) selecting backcross progeny plants that have the desired trait and physiological and morphological characteristics of seashore paspalum variety ‘UGA 17-726’ to produce selected backcross progeny plants; and (e) repeating steps (c) and (d) one or more times in succession to produce selected second or higher backcross progeny plants that include the desired trait and the physiological and morphological characteristics of turfgrass variety ‘UGA 17-726’ when grown in the same environmental conditions. In some embodiments, the single locus confers a desirable trait, such as herbicide tolerance, resistance to an insect, resistance to a bacterial disease, resistance to a viral disease, resistance to a fungal disease, resistance to a nematode, resistance to a pest, male sterility, site-specific recombination; and/or abiotic stress tolerance (such as tolerance to drought, heat, low or high soil pH level, and/or salt).

Methods of producing a seashore paspalum plant derived from the new variety ‘UGA 17-726’, such as an inbred seashore paspalum plant, are provided. In particular examples the method includes (a) preparing a progeny plant derived from the new variety ‘UGA 17-726’ by crossing a plant of ‘UGA 17-726’ with a seashore paspalum plant of a second variety; and (b) crossing the progeny plant with itself or a second plant to produce a progeny plant of a subsequent generation which is derived from a plant of the new variety ‘UGA 17-726’. In some embodiments, the method further includes (c) growing a progeny plant of a subsequent generation from said seed and crossing the progeny plant of a subsequent generation with itself or a second plant; and (d) repeating steps (b) and (c) for at least 2 additional generations (such as at least 3, at least 5, or at least 10 additional generations, such as 2, 3, 4, 5, 6, 7, 8, 9, 10, 11, 12, 13, 14, 15, 16, 17, 18, 19 or 20 additional generations) with sufficient inbreeding to produce an inbred seashore paspalum plant derived from the new variety ‘UGA 17-726’. In other examples, the method includes (a) crossing a seashore paspalum plant derived from the new variety ‘UGA 17-726’ with itself or another seashore paspalum plant to yield additional variety ‘UGA 17-726’-derived progeny seed; (b) growing the progeny seed of (a) under plant growth conditions, to yield additional seashore paspalum variety ‘UGA 17-726’-derived turfgrass plants; and (c) repeating the crossing and growing steps of (a) and (b) from 0 to 7 times (such as 0 to 4 or 1 to 5 times, such as 0, 1, 2, 3, 4, 5, 6, or 7 times) to generate further seashore paspalum variety ‘UGA 17-726’-derived seashore paspalum plants.

Methods are provided for developing a new turfgrass plant using the new ‘UGA 17-726’ variety. For example, the methods can include using ‘UGA 17-726’ plants or parts thereof as a source of breeding material in plant breeding techniques, such as recurrent selection, mass selection, bulk selection, backcrossing, pedigree breeding, genetic marker-assisted selection and genetic transformation. In some examples, a plant of the new seashore paspalum variety ‘UGA 17-726’ is used as the male or female parent.

The disclosure provides a first generation (F₁) hybrid seashore paspalum seed produced by crossing a plant of the new seashore paspalum variety ‘UGA 17-726’ to a second seashore paspalum plant. In some embodiments, the F₁ hybrid plant is grown from the hybrid seed produced by crossing the new seashore paspalum variety ‘UGA 17-726’ to a second seashore paspalum plant.

Methods of producing hybrid seashore paspalum seeds are also provided. In one example the method includes crossing the new variety ‘UGA 17-726’ to a second, distinct seashore paspalum plant which is non-isogenic to the new variety ‘UGA 17-726’. In some examples, the method includes cultivating plants grown from seeds of the new variety ‘UGA 17-726’ and cultivating seashore paspalum plants grown from seeds of a second, distinct seashore paspalum plant, until the plants bear flowers. A flower on one of the two plants is cross pollinated with the pollen of the other plant, and the seeds resulting from such a cross are harvested.

The disclosure also provides seashore paspalum plants and parts thereof produced by any of the methods disclosed herein. Thus, provided herein are plants of seashore paspalum variety ‘UGA 17-726’ that further include a single locus conversion, such as one or more desired traits, for example produced by backcrossing or genetic transformation. In some embodiments, the seashore paspalum plants produced by the disclosed methods include at least two, at least three, or at least four of the traits of the new seashore paspalum variety ‘UGA 17-726’ as described herein, (see, e.g., Examples), such as but not limited to ACCase-inhibiting herbicide resistance, high salt tolerance, and the like. In embodiments, at least 1 of the traits is ACCase-inhibiting herbicide resistance.

One method of identifying a superior plant is to observe its performance relative to other experimental plants and to one or more widely grown standard varieties. Single observations can be generally inconclusive, while replicated observations provide a better estimate of genetic worth.

Plant breeding can result in new, unique and superior seashore paspalum varieties and hybrids from ‘UGA 17-726’. Two or more parental lines can be selected (such as ‘UGA 17-726’ as one of the lines), followed by repeated selfing and selection, producing many new genetic combinations. Each year, the germplasm to advance to the next generation is selected. This germplasm is grown under unique and different geographical, climatic and soil conditions, and further selections are then made, during and at the end of the growing season.

In some examples, new seashore paspalum varieties developed from ‘UGA 17-726’ (such as F₁, F₂, F₃, F₄, F₅, F₆, F₇ F₈, F₉, or F₁₀ progeny, or even later progeny) are exposed to sethoxyrim of a like compound, they are resistant to such herbicides.

The development of new seashore paspalum varieties from ‘UGA 17-726’ involves the development and selection of seashore paspalum varieties, the crossing of these varieties and selection of progeny from the superior hybrid crosses. A hybrid seed is produced by manual crosses between selected male-fertile parents or by using male sterility systems. Hybrids can be identified by using certain single locus traits such as pod color, flower color, seed color, or pubescence color, which indicate that the seed is truly a hybrid. Additional data on parental lines as well as the phenotype of the hybrid can influence a decision whether to continue with the specific hybrid cross.

Pedigree breeding and recurrent selection breeding methods can be used to develop varieties from breeding populations. Breeding programs combine desirable traits from two or more varieties or various broad-based sources into breeding pools from which varieties are developed by selfing and selection of desired phenotypes. Pedigree breeding is commonly used for the improvement of self-pollinating crops. Two parents (e.g., wherein one of the parents is ‘UGA 17-726’) which possess favorable, complementary traits are crossed to produce an F₁. An F₂ population is produced by allowing one or several F₁ seashore paspalum plants to self-pollinate. Selection of the best or most preferred individuals usually begins in the F₂ population (or later depending upon the breeding objectives); then, beginning in the F₃, the best or most preferred individuals in the best families can be selected. Replicated testing of families can begin in the F₃ or F₄ generation to improve the effectiveness of selection for traits with low heritability. At an advanced stage of inbreeding (e.g., F₆ and F₇), the best lines or mixtures of phenotypically similar lines can begin replicated testing for potential commercial release as new varieties.

Mass and recurrent selections can be used to improve populations of either self- or cross-pollinating crops. A genetically variable population of heterozygous individuals is either identified or created by intercrossing several different parents. The best or most preferred plants are selected based on individual superiority, outstanding progeny, or excellent combining ability. The selected plants are intercrossed to produce a new population in which further cycles of selection are continued.

Backcross breeding can be used to transfer genetic loci for simply inherited, highly heritable traits into a desirable homozygous variety which is the recurrent parent (e.g., ‘UGA 17-726’). The source of the trait to be transferred is called the donor or nonrecurrent parent. The resulting plant is expected to have the attributes of the recurrent parent (e.g., variety) and the desirable trait transferred from the donor parent. After the initial cross, individuals possessing the phenotype of the donor parent are selected and repeatedly crossed (backcrossed) to the recurrent parent. The resulting plant is typically expected to have the attributes of the recurrent parent (e.g., variety) and the desirable trait transferred from the donor parent.

The single-seed descent procedure can refer to planting a segregating population, harvesting a sample of one seed per plant, and using the one-seed sample to plant the next generation. When the population has been advanced from the F₂ to the desired level of inbreeding, the plants from which lines are derived will each trace to different F₂ individuals. The number of plants in a population declines each generation due to failure of some seeds to germinate or some plants to produce at least one seed. As a result, not all of the F₂ plants originally sampled in the population are represented by a progeny when generation advance is completed.

In a multiple-seed procedure, one or more pods from each plant in a population are commonly harvested and threshed together to form a bulk. Part of the bulk is used to plant the next generation and part is put in reserve. The procedure has been referred to as modified single-seed descent or the pod-bulk technique. The multiple-seed procedure has been used to save labor at harvest. It is faster to thresh pods with a machine than to remove one seed from each by hand for the single-seed procedure. The multiple-seed procedure also makes it possible to plant the same number of seeds of a population each generation of inbreeding. Sufficient numbers of seeds are harvested to make up for those plants that did not germinate or produce seed.

Descriptions of other breeding methods commonly used for different traits and crops can be found in one of several reference books (e.g., Allard. 1960. Principles of plant breeding. Davis, California: John Wiley & Sons, NY, University of California, pp. 50-98; Simmonds. 1979. Principles of crop improvement. New York: Longman, Inc., pp. 369-399; Sneep and Hendriksen. 1979. “Plant breeding perspectives.” Wageningen (ed.), Center for Agricultural Publishing and Documentation; Fehr. 1987. “Principles of variety development.” Theory and Technique (Vol. 1).

### Breeding Seashore Paspalum Variety ‘UGA 17-726’

Methods for crossing the new seashore paspalum variety ‘UGA 17-726’ with itself or a second plant are provided, as are the seeds and plants produced by such methods. Such methods can be used for propagation of the new turfgrass variety ‘UGA 17-726’, or progeny thereof, can be used to produce hybrid seashore paspalum seeds and the plants grown therefrom. Hybrid seashore paspalum plants can be used, for example, in the commercial cultivation of turfgrasses or in breeding programs for the production of novel turfgrass varieties. A hybrid plant can also be used as a recurrent parent at any given stage in a backcrossing protocol during the production of a single locus conversion (for example introduction of one or more desirable traits) of the new seashore paspalum variety ‘UGA 17-726’.

Methods of producing seashore paspalum plants and/or seed are provided. Such a method can include crossing the new variety ‘UGA 17-726’ with itself or a second seashore paspalum plant and harvesting a resulting seed, such as an F₁ hybrid seed. The resulting plant can be grown, resulting in a seashore paspalum plant or part thereof.

In one example methods of producing an inbred seashore paspalum plant derived from variety ‘UGA 17-726’ are provided. In one example such methods include (a) preparing a progeny plant derived from variety ‘UGA 17-726’ by crossing a plant of the variety ‘UGA 17-726’ with a seashore paspalum plant of a second variety; (b) crossing the progeny plant with itself or a second plant to produce a seed of a progeny plant of a subsequent generation; (c) growing the progeny plant of the subsequent generation from said seed and crossing the progeny plant of the subsequent generation with itself or a second plant; and (d) repeating steps (b) and (c) for an additional at least 2 generations (such as at least 3, at least 4, at least 5, at least 6, at least 7, at least 8 at least 9, at least 10, at least 15 or at least 20, such as 2 to 10, 3 to 10, or 3 to 15 generations) with sufficient inbreeding to produce an inbred seashore paspalum plant derived from the variety ‘UGA 17-726’.

The second plant crossed with the new seashore paspalum variety ‘UGA 17-726’ for the purpose of developing novel turfgrass varieties, is typically a plant which either themselves exhibit one or more desirable characteristics or which exhibit one or more desired characteristic(s) when in hybrid combination. In one example, the second seashore paspalum plant is transgenic. Exemplary desired characteristics include, but are not limited to, one or more of: increased seed yield, lodging resistance, emergence, increased seedling vigor, modified maturity date, desired plant height, herbicide tolerance, drought tolerance, heat tolerance, low or high soil pH level tolerance, salt tolerance, resistance to an insect, resistance to a bacterial disease, resistance to a viral disease, resistance to a fungal disease, resistance to a nematode, resistance to a pest, male sterility, site-specific recombination; abiotic stress tolerance; modified phosphorus content, modified antioxidant content; modified essential seed amino acid content, modified fatty acid content, modified carbohydrate content, and modified seed yield.

When the new seashore paspalum variety ‘UGA 17-726’ is crossed with another different variety, first generation (F₁) seashore paspalum progeny are produced. The hybrid progeny are produced regardless of characteristics of the two varieties produced. As such, an F₁ hybrid plant can be produced by crossing ‘UGA 17-726’ with any second seashore paspalum plant. The second seashore paspalum plant can be genetically homogeneous (e.g., inbred) or can itself be a hybrid. Therefore, the disclosure provides any F₁ hybrid seashore paspalum plant produced by crossing the new variety ‘UGA 17-726’ with a second seashore paspalum plant (such as a transgenic plant having one or more genes that confer to the plant one or more desired characteristics).

Seashore paspalum plants can be crossed by either natural or mechanical techniques. Natural pollination occurs in grass plants either by self-pollination or natural cross pollination, which typically is aided by wind. In either natural or artificial crosses, flowering time can be a consideration.

### Turfgrass Plants Having One or More Desired Heritable Traits

Seashore paspalum varieties can also be developed from more than two parents, for example using modified backcrossing, which uses different recurrent parents during the backcrossing. Modified backcrossing can be used to replace the original recurrent parent with a variety having certain more desirable characteristics, or multiple parents can be used to obtain different desirable characteristics from each.

Useful or desirable traits can be introduced by backcrossing, as well as directly into a plant by genetic transformation methods. Genetic transformation can therefore be used to insert a selected transgene into the ‘UGA 17-726’ variety or can, alternatively, be used for the preparation of transgenes which can be introduced by backcrossing. Thus, the disclosure provides methods of producing a plant of variety ‘UGA 17-726’ that includes one or more added desired traits, for example that include introducing a transgene(s) conferring the one or more desired traits into a plant of variety ‘UGA 17-726’ (for example by transformation with a transgene that confers upon the seashore paspalum plant the desired trait), thereby producing a plant of seashore paspalum variety ‘UGA 17-726’ that includes the one or more added desired traits.

Methods for the transformation of many economically important plants, including turfgrasses, are known. Methods for introducing a desired nucleic acid molecule (e.g., transgene), such as DNA, RNA, or inhibitory RNAs, are known, and the disclosure is not limited to particular methods. Exemplary techniques which can be employed for the genetic transformation of seashore paspalum include, but are not limited to, electroporation, microprojectile bombardment, Agrobacterium-mediated transformation direct DNA uptake by protoplasts, sonication of target cells, liposome and spheroplast fusion, CaCl2 precipitation, polyvinyl alcohol, or poly-L-ornithine.

To effect transformation by electroporation, friable tissues, such as a suspension culture of cells or embryogenic callus, can be used. Alternatively, immature embryos or other organized tissue can be transformed directly. In this technique, the cell walls of target cells can be partially degraded by exposing them to pectin-degrading enzymes (pectolyases) or mechanically wound tissues in a controlled manner.

Protoplasts can also be employed for electroporation transformation of plants (Bates. 1994. Mol. Biotechnol. 2(2):135-145; Lazzeri. 1995. Methods Mol. Biol. 49:95-106). For example, the generation of transgenic seashore paspalum plants by electroporation of cotyledon-derived protoplasts and whole cells and tissues has been described (Donn et al., In Abstracts of VIIth International Congress on Plant Cell and Tissue Culture IAPTC, A2-38, p. 53 (1990); D'Halluin et al., Plant Cell, 4:1495-1505 (1992); and Spencer et al., Plant Mol. Biol., 24:51-61 (1994)). In microprojectile bombardment, particles (such as those comprised of tungsten, platinum, or gold) are coated with nucleic acids and delivered into cells by a propelling force. For the bombardment, cells in suspension are concentrated on filters or solid culture medium. Alternatively, immature embryos or other target cells can be arranged on solid culture medium. The cells to be bombarded are positioned at an appropriate distance below the macroprojectile stopping plate. An exemplary method for delivering DNA into plant cells by acceleration is the Biolistics Particle Delivery System, which can be used to propel particles coated with DNA or cells through a screen, such as a stainless steel or Nytex screen, onto a surface covered with target seashore paspalum cells. The screen disperses the particles so that they are not delivered to the recipient cells in large aggregates. A screen intervening between the projectile apparatus and the cells to be bombarded can reduce the size of projectiles aggregate and contribute to a higher frequency of transformation by reducing the damage inflicted on the recipient cells by projectiles that are too large. Microprojectile bombardment methods can be used to transform turfgrasses, as described, for example, in U.S. Pat. No. 5,322,783.

Agrobacterium-mediated transfer can be used to introduce gene loci into plant cells. DNA can be introduced into whole plant tissues, thereby bypassing the need for regeneration of an intact plant from a protoplast. Agrobacterium transformation vectors are capable of replication in E. coli as well as Agrobacterium, allowing for convenient manipulations (Klee et al. 1985. Bio. Tech. 3(7):637-342). Moreover, vectors for Agrobacterium-mediated gene transfer have improved the arrangement of genes and restriction sites in the vectors to facilitate the construction of vectors capable of expressing various polypeptide coding genes. Such vectors have convenient multi-linker regions flanked by a promoter and a polyadenylation site for direct expression of inserted polypeptide coding genes. Additionally, Agrobacterium containing both armed and disarmed Ti genes can be used for transformation. The use of Agrobacterium-mediated plant integrating vectors to introduce DNA into plant cells is known (e.g., Fraley et al. 1985. Bio. Tech. 3(7):629-635; U.S. Pat. No. 5,563,055), and its use for turfgrass transformation has been described (Chee and Slightom. 1995. Methods Mol. Biol. 44:101-119; U.S. Pat. No. 5,569,834). Briefly, plant tissue (often leaves) is cut into small pieces, e.g., 10 mm×10 mm, and soaked for 10 minutes in a fluid containing suspended Agrobacterium. Some cells along the cut will be transformed by the bacterium, which inserts its DNA into the cell, which is placed on selectable rooting and shooting media, allowing the plants to regrow. Some plants can be transformed just by dipping the flowers into suspension of Agrobacterium and then planting the seeds in a selective medium.

Transformation of plant protoplasts can also be achieved using methods based on calcium phosphate precipitation, polyethylene glycol treatment, electroporation, and combinations of these treatments (e.g., Potrykus et al. 1985. Mol. Gen. Genet. 199(2):169-177; Omirulleh et al. 1993. Plant Mol. Biol. 21(3):415-428; Fromm et al. 1986. Nature. 319(6056):791-739; Uchimiya et al. 1986. Mol. Gen. Genet. 204(2):207-207; Marcotte et al. 1988. Nature 335(6189):454-457). The ability to regenerate seashore paspalum plants from protoplasts makes these techniques applicable to turfgrasses.

In one example, such methods can also be used to introduce transgenes for the production of proteins in transgenic turfgrasses. The resulting produced protein can be harvested from the transgenic turfgrass. The transgene can be harvested from the transgenic plants that are originated or are descended from the new seashore paspalum variety ‘UGA 17-726’, a seed of ‘UGA 17-726’ or a hybrid progeny of ‘UGA 17-726’.

Numerous different genes are known and can be introduced into a seashore paspalum plant ‘UGA 17-726’ or progeny thereof. Non-limiting examples of particular genes and corresponding phenotypes that can be chosen for introduction into a seashore paspalum plant are provided herein.

### Herbicide Resistance

Numerous herbicide resistance genes are known and can be used with the methods and plants provided herein. In particular examples, a herbicide resistance gene confers tolerance to an herbicide comprising glyphosate, sulfonylurea, imidazalinone, dicamba, glufosinate, phenoxy proprionic acid, cyclohexone, triazine, benzonitrile, broxynil, L-phosphinothricin, cyclohexanedione, chlorophenoxy acetic acid, or combinations thereof.

In one example the herbicide resistance gene is a gene that confers resistance to an herbicide that inhibits the growing point or meristem, such as an imidazalinone or a sulfonylurea. Exemplary genes in this category code for mutant ALS and AHAS enzyme as described, for example, by Lee et al. (1988. Embryo J. 7:1241-8) and Miki et al. (1990. Theoret. Appl. Genet. 80:449-458).

Resistance genes for glyphosate (e.g., resistance conferred by mutant 5-enolpyruvl-3 phosphikimate synthase (EPSP) and aroA genes, respectively) and other phosphono compounds such as glufosinate (phosphinothricin acetyl transferase (PAT) and Streptomyces hygroscopicus phosphinothricin-acetyl transferase (bar) genes), pyridinoxy or phenoxy proprionic acids, and cyclohexanediones (ACCase inhibitor-encoding genes), can be used (e.g., see U.S. Pat. Nos. 4,940,835; 5,627,061; 6,566,587, 6,338,961, 6,248,876, 6,040,497, 5,804,425, 5,633,435, 5,145,783, 4,971,908, 5,312,910, 5,188,642, 4,940,835, 5,866,775, 6,225,114, 6,130,366, 5,310,667, 4,535,060, 4,769,061, 5,633,448, 5,510,471, RE 36,449, RE 37,287, 5,491,288, 5,776,760, 5,463,175, 7,462,481; and International Publications EP1173580, WO 01/66704, EP1173581, and EP1173582). Examples of specific EPSPS transformation events conferring glyphosate resistance are described, for example, in U.S. Pat. No. 6,040,497.

DNA molecules encoding a mutant aroA gene are known (e.g., ATCC accession number 39256 and U.S. Pat. No. 4,769,061), as are sequences for glutamine synthetase genes, which confer resistance to herbicides such as L-phosphinothricin (e.g., U.S. Pat. No. 4,975,374), phosphinothricin-acetyltransferase (e.g., U.S. Pat. No. 5,879,903). DeGree F. et al. (1989. Bio/Technology 61-64) describe the production of transgenic plants that express chimeric bar genes coding for phosphinothricin acetyl transferase activity. Exemplary genes conferring resistance to phenoxy propionic acids and cyclohexones, such as sethoxydim and haloxyfop are the Acct-S1, Accl-S2 and Acct-S3 genes described by Marshall et al. (1992. Theor Appl Genet. 83:435-442).

Genes conferring resistance to an herbicide that inhibits photosynthesis are also known, such as, a triazine (psbA and gs+ genes) and a benzonitrile (nitrilase gene) (see Przibilla et al., 1991. Plant Cell. 3:169-174). Nucleotide sequences for nitrilase genes are disclosed in U.S. Pat. No. 4,810,648, and DNA molecules containing these genes are available under ATCC Accession Nos. 53435, 67441, and 67442. Cloning and expression of DNA coding for a glutathione S-transferase is described by Hayes et al. (1992. Biochem. J. 285:173).

U.S. Patent Publication No: 20030135879 describes dicamba monooxygenase (DMO) from Pseuodmonas maltophilia, which is involved in the conversion of a herbicidal form of the herbicide dicamba to a non-toxic 3,6-dichlorosalicylic acid and thus can be used for producing plants tolerant to this herbicide.

The metabolism of chlorophenoxyacetic acids, such as, for example 2,4-D herbicide, is known. Genes or plasmids that contribute to the metabolism of such compounds are described, for example, by Muller et al. (2006. Appl. Environ. Microbiol. 72(7):4853-4861), Don and Pemberton (1981. J Bacteriol 145(2):681-686), Don et al. (1985. J Bacteriol 161(1):85-90) and Evans et al. (1971. Biochem J 122(4):543-551).

Acetohydroxy acid synthase, which has been found to make plants that express this enzyme resistant to multiple types of herbicides, has been introduced into a variety of plants. See, Hattori et al., Mol. Gen. Genet., 246:419 (1995). Other genes that confer tolerance to herbicides include a gene encoding a chimeric protein of rat cytochrome P4507A1 and yeast NADPH-cytochrome P450 oxidoreductase (Shiota et al., Plant Physiol., 106:17 (1994)); genes for glutathione reductase and superoxide dismutase (Aono, et al., Plant Cell Physiol., 36:1687 (1995)); and genes for various phosphotransferases (Datta, et al., Plant Mol. Biol., 20:619 (1992)).

Protoporphyrinogen oxidase (protox) is necessary for the production of chlorophyll, which is necessary for all plant survival. The protox enzyme serves as the target for a variety of herbicidal compounds. These herbicides also inhibit growth of all the different species of plants present, causing their total destruction. The development of plants containing altered protox activity which are resistant to these herbicides are described in U.S. Pat. Nos. 6,288,306, 6,282,837, 5,767,373, and International Publication WO 01/12825.

Any of the above listed herbicide genes can be introduced into the disclosed ‘UGA 17-726’ through a variety of means including but not limited to transformation and crossing.

### Male Sterility

Genetic male sterility can increase the efficiency with which hybrids are made, in that it can eliminate the need to physically emasculate the seashore paspalum plant used as a female in a given cross (Brim and Stuber. 1973. Crop Sci. 13:528-530). Herbicide-inducible male sterility systems are known (e.g., U.S. Pat. No. 6,762,344).

Where use of male-sterility systems is desired, it can be beneficial to also utilize one or more male-fertility restorer genes. For example, where cytoplasmic male sterility (CMS) is used, hybrid seed production involves three inbred lines: (1) a cytoplasmically male-sterile line having a CMS cytoplasm; (2) a fertile inbred with normal cytoplasm, which is isogenic with the CMS line for nuclear genes (“maintainer line”); and (3) a distinct, fertile inbred with normal cytoplasm, carrying a fertility restoring gene (“restorer” line). The CMS line is propagated by pollination with the maintainer line, with all of the progeny being male sterile, as the CMS cytoplasm is derived from the female parent. These male sterile plants can then be efficiently employed as the female parent in hybrid crosses with the restorer line, without the need for physical emasculation of the male reproductive parts of the female parent.

The presence of a male-fertility restorer gene results in the production of fully fertile F₁ hybrid progeny. If no restorer gene is present in the male parent, male-sterile hybrids are obtained. Such hybrids are useful where the vegetative tissue of the seashore paspalum plant is utilized. However, in many cases, the seeds are considered to be a valuable portion of the crop, thus, it is desirable to restore the fertility of the hybrids in these crops. Therefore, the disclosure provides plants of the new seashore paspalum variety ‘UGA 17-726’ comprising a genetic locus capable of restoring male fertility in an otherwise male-sterile plant. Examples of male-sterility genes and corresponding restorers which can be employed are known (see, e.g., U.S. Pat. Nos. 5,530,191 and 5,684,242).

There are several methods of conferring genetic male sterility available, such as multiple mutant genes at separate locations within the genome that confer male sterility, as disclosed in U.S. Pat. Nos. 4,654,465 and 4,727,219 and chromosomal translocations as described in U.S. Pat. Nos. 3,861,709 and 3,710,511. In addition to these methods, U.S. Pat. No. 5,432,068, describes a system of nuclear male sterility which includes: identifying a gene which is critical to male fertility; silencing this native gene which is critical to male fertility; removing the native promoter from the essential male fertility gene and replacing it with an inducible promoter; inserting this genetically engineered gene back into the plant; and thus creating a plant that is male sterile because the inducible promoter is not “on” resulting in the male fertility gene not being transcribed. Fertility is restored by inducing, or turning “on,” the promoter, which in turn allows the gene that confers male fertility to be transcribed.

Introduction of a deacetylase gene under the control of a tapetum-specific promoter and with the application of the chemical N—Ac—PPT. See WO 01/29237.

Introduction of various stamen-specific promoters. See WO 92/13956 and WO 92/13957.

Introduction of the barnase and the barstar genes. See, Paul et al., Plant Mol. Biol., 19:611-622 (1992).

For additional examples of nuclear male and female sterility systems and genes, see also, U.S. Pat. Nos. 5,859,341, 6,297,426, 5,478,369, 5,824,524, 5,850,014, and 6,265,640.

Any of the above-listed male sterility genes can be introduced into ‘UGA 17-726’ through a variety of means including, but not limited to, transformation and crossing.

Genes that Create a Site for Site Specific DNA Integration

This includes the introduction of FRT sites that may be used in the FLP/FRT system and/or Lox sites that may be used in the Cre/Loxp system. See, for example, Lyznik, et al., Site-Specific Recombination for Genetic Engineering in Plants, Plant Cell Rep, 21:925-932 (2003) and WO 99/25821, which are hereby incorporated by reference. Other systems that may be used include the Gin recombinase of phage Mu (Maeser, et al. (1991); Vicki Chandler, The Maize Handbook, Ch. 118 (Springer-Verlag 1994)); the Pin recombinase of E. coli (Enomoto, et al. (1983)); and the R/RS system of the pSR1 plasmid (Araki, et al. (1992)).

Any of the above-listed sites can be introduced into ‘UGA 17-726’ through a variety of means including, but not limited to, transformation and crossing.

Genes that Affect Abiotic Stress Resistance

Genes that affect abiotic stress resistance (including but not limited to flowering, pod and seed development, enhancement of nitrogen utilization efficiency, altered nitrogen responsiveness, drought resistance or tolerance, cold resistance or tolerance, and salt resistance or tolerance) and increased yield under stress. For example, see: WO 00/73475 where water use efficiency is altered through alteration of malate; U.S. Pat. Nos. 5,892,009, 5,965,705, 5,929,305, 5,891,859, 6,417,428, 6,664,446, 6,706,866, 6,717,034, 6,801,104, WO 2000/060089, WO 2001/026459, WO 2001/035725, WO 2001/034726, WO 2001/035727, WO 2001/036444, WO 2001/036597, WO 2001/036598, WO 2002/015675, WO 2002/017430, WO 2002/077185, WO 2002/079403, WO 2003/013227, WO 2003/013228, WO 2003/014327, WO 2004/031349, WO 2004/076638, WO 98/09521, and WO 99/38977 describing genes, including CBF genes and transcription factors effective in mitigating the negative effects of freezing, high salinity, and drought on plants, as well as conferring other positive effects on plant phenotype; U.S. Publ. No. 2004/0148654 and WO 01/36596, where abscisic acid is altered in plants resulting in improved plant phenotype, such as increased yield and/or increased tolerance to abiotic stress; WO 2000/006341, WO 04/090143, U.S. Pat. Nos. 7,531,723, and 6,992,237, where cytokinin expression is modified resulting in plants with increased stress tolerance, such as drought tolerance, and/or increased yield. See also, WO 02/02776, WO 2003/052063, JP 2002281975, U.S. Pat. No. 6,084,153, WO 01/64898, and U.S. Pat. Nos. 6,177,275 and 6,107,547 (enhancement of nitrogen utilization and altered nitrogen responsiveness). For ethylene alteration, see, U.S. Publ. Nos. 2004/0128719, 2003/0166197, and WO 2000/32761. For plant transcription factors or transcriptional regulators of abiotic stress, see, e.g., U.S. Publ. Nos. 2004/0098764 or 2004/0078852.

Other genes and transcription factors that affect plant growth and agronomic traits, such as yield, flowering, plant growth, and/or plant structure, can be introduced or introgressed into plants. See, e.g., WO 97/49811 (LHY), WO 98/56918 (ESD4), WO 97/10339, U.S. Pat. No. 6,573,430 (TFL), 6,713,663 (FT), 6,794,560, 6,307,126 (GAI), WO 96/14414 (CON), WO 96/38560, WO 01/21822 (VRN1), WO 00/44918 (VRN2), WO 99/49064 (GI), WO 00/46358 (FR1), WO 97/29123, WO 99/09174 (D8 and Rht), WO 2004/076638, and WO 004/031349 (transcription factors).

Any of the above-listed sites can be introduced into ‘UGA 17-726’ through a variety of means including, but not limited to, transformation and crossing.

### Tissue Cultures and In Vitro Regeneration of Turfgrass Plants

A tissue culture includes isolated cells of the same or a different type or a collection of such cells organized into parts of a plant. Exemplary types of tissue cultures include protoplasts, calli and plant cells that are intact in plants or parts of plants, such as embryos, pollen, flowers, leaves, roots, root tips, anthers, meristematic cells, pistil, seed, pod, petiole, stein, ovule, cotyledon, hypocotyl, shoot, stem, and the like. In a particular example, the tissue culture includes embryos, protoplasts, meristematic cells, pollen, leaves or anthers of the new seashore paspalum variety ‘UGA 17-726’. Also provided are seashore paspalum plants regenerated from such tissue cultures, wherein the regenerated seashore paspalum plant expresses the physiological and morphological characteristics of the seashore paspalum variety ‘UGA 17-726’.

Exemplary methods for preparing and maintaining plant tissue culture are described in.

U.S. Pat. Nos. 5,959,185, 5,973,234, and 5,977,445.

### Example 1

### Genotyping of 1781 Leu Mutants Induced by Tissue Culture of Paspalum Cultivar Mauna Kea: Evidence of Genetic Heritability of the 1781 Leu Mutation

DNA samples were extracted from Mauna Kea the original source of explant material as well as from 23 tissue cultural derived lines. One non-mutant plant regenerated from tissue culture was included as a tissue culture control (TCC). Three SSR DNA markers were used to genotype the 22 herbicide resistant mutants and 2 controls. The parental line Mauna Kea and TCC Mauna Kea showed identical profiles in for all 3 SSR primers. However, DNA banding patterns differed from the parental line in 9 of the 23 lines derived from tissue culture (Table 1, Fin. 1)

TABLE 1
Summary of genotyping results of Maunea Kea (parent line) and 23 lines
derived via tissue culture during selection for sethoxydim resistance

| | | | | | DNA Analysis | | |
|---|---|---|---|---|---|---|---|
| | | | Cell | | (SSR)² | | |
| Tube | Plant Description | Genotype | Line | 1781Leu¹ | Pv3 | Pv35 | Pv51 |
| S1 | Parent Genotype (susceptible) | Maunea | | − | ✓ | ✓ | ✓ |
| | | Kea | | | | | |
| S2 | Tissue Culture Control | Maunea | | − | ✓ | ✓ | ✓ |
| | (susceptible) | Kea TCC | | | | | |
| S3 | Plant D from SR 11 callus | SR 11D | 11 | + | ✓ | ✓ | ✓ |
| S4 | Clone 1 of plant HH from SR | SR11 HH- | 11 | + | X | ✓ | ✓ |
| | 11 callus | 1 | | | | | |
| S5 | Clone 2 of plant HH from SR | SR11 HH- | 11 | + | X | X | ✓ |
| | 11 callus | 2 | | | | | |
| S6 | Clone 2 of plant HH from SR | SR11 HH- | 11 | + | ✓ | X | ✓ |
| | 11 callus | 3 | | | | | |
| S7 | Plant 102 from SR 11 callus | SR11-102 | 11 | + | X | X | ✓ |
| S8 | Plant 214 from SR 11 callus | SR11-214 | 11 | + | ✓ | ✓ | ✓ |
| S9 | Vegetative Clone 1 of plant 14 | SR31.14- | 31 | + | ✓ | ✓ | ✓ |
| | from SR 31 callus | 1 | | | | | |
| S10 | Vegetative Clone 2 of plant 14 | SR31.14- | 31 | + | ✓ | ✓ | ✓ |
| | from SR 31 callus | 2 | | | | | |
| S11 | Vegetative Clone 3 of plant 14 | SR31.14- | 31 | + | ✓ | ✓ | ✓ |
| | from SR 31 callus | 3 | | | | | |
| S12 | Vegetative Clone 1 of plant 15 | SR31.15- | 31 | + | ✓ | ✓ | ✓ |
| | from SR 31 callus | 1 | | | | | |
| S13 | Vegetative Clone 2 of plant 15 | SR31.15- | 31 | + | ✓ | ✓ | ✓ |
| | from SR 31 callus | 2 | | | | | |
| S14 | Vegetative Clone 3 of plant 15 | SR31.15- | 31 | + | ✓ | ✓ | ✓ |
| | from SR 31 callus | 3 | | | | | |
| S15 | Plant 35 from SR 31 callus | SR31.35 | 31 | + | ✓ | ✓ | ✓ |
| S16 | Plant 36 from SR 31 callus | SR31.36 | 31 | + | ✓ | ✓ | ✓ |
| S17 | Plant 54 from SR 31 callus | SR31.54 | 31 | + | X | ✓ | X |
| S18 | Plant 55 from SR 31 callus | SR31.55 | 31 | + | X | ✓ | X |
| S19 | Plant 77 from SR 31 callus | SR31.77 | 31 | + | ✓ | ✓ | ✓ |
| S20 | Plant 79 from SR 31 callus | SR31.79 | 31 | + | X | X | X |
| S21 | Plant 96 from SR 31 callus | SR31.96 | 31 | + | X | X | ✓ |
| S22 | Plant 108 from SR 31 callus | SR31.108 | 31 | + | X | ✓ | X |
| S23 | Plant 110 from SR 31 callus | SR31.110 | 31 | + | ✓ | ✓ | ✓ |
| S24 | Plant 129 from SR 31 callus | SR31.129 | 31 | + | ✓ | ✓ | ✓ |

1. ‘−’ indicates normal “wild type” (susceptible) and ‘+’ indicates presence of mutation conferring sethoxydim resistance.
2. ‘✓’ indicates that the line is identical to the parental line, Maunea Kea and ‘X’ indicates differences in banding patterns from the parental line.

### Example 2

### Restoration of Fertility

Following years of failed attempts to utilize this novel material for breeding new varieties, one plant, SR 31.15, was identified with good vigor and the ability to produce flowers, however, repeated hand pollinations still resulted in no viable seed. This plant was genetically stabilized using internodal tissue to regenerate a genetically stable plant.

In an attempt to overcome the inherent sterility of this tissue culture derived line, the mutant sethoxydim resistant line, SR31.15 (Rr), heterozygous for the 1781 Leu mutation, was crossed with the sethoxydim susceptible commercial cultivar ‘Sealsle 1’ (rr) and embryo rescue used to recover viable seedlings. SNP primers were used to identify plants carrying the mutant allele (1781 Leu). Six embryo-derived offspring of this cross were found to be heterozygous (Rr) for the mutant allele.

### Example 3

### Evidence of Stable Inheritance of the Novel Trait

Two of these six lines, SR31.15.1 (Rr) and SR31.15.5 (Rr), were crossed during the spring of 2014 using hand pollination and successfully produced viable seed indicating that fertility had been restored to the embryo rescue derived lines. Eight progenies from this cross were successfully grown into plants from this cross pollination. DNA was successfully extracted from seven of these progeny and the presence of the 1781 Leu mutation was tested using SNP primers as described above. The expected ratio for this cross assuming a simply inherited dominant trait is 1:3:1. Our observed results indicated a genotypic ratio of 1 RR:5 Rr:1 rr and was not statistically different from the expected ratio according to a Chi-square statistical test. The original mutant line SR31.15 was heterozygous for the trait (Rr) and we have now produced a plant homozygous for the trait (RR) using sexual crosses. This is clear evidence of the presence of a novel and simply inherited dominant trait in this species.

### Example 4

### Summary of Breeding History of UGA 17-726

‘Mauna Kea’ Immature inflorescence were used as explants for tissue culture. Callus induction and increase were performed prior to in vitro selection for resistance to sethoxydim. SR 11 and SR 31 cell lines were heterozygous for 1781 Leu mutation and resistant to sethoxydim and were used to regenerate individual plants. Approximately 2000 individual plants were generated and evaluated for breeding potential. Most plants lacked vigor, and all were non-reproductive.

SR 31.15 (Rr) was selected as vigorous and flowering, but produced no viable seed when crossed. SR31.15 (Rr) x ‘Sealsle 1’ (rr) hand pollination crosses were followed by embryo rescue and tissue culture of immature embryos. SR31.15.1-SR31.15.6 led to 6 viable plants (Rr) expressing the 1781 Leu mutation.

SR31.15.1 (Rr) x SR31.15.5 (Rr) hand pollinated cross of 2 embryo rescued lines was heterozygous for 1781 Leu). 7 progeny showed 1 RR:5 Rr:1 rr segregation for 1781 Leu mutation. The homozygous line was designated as SR14-1E (RR).

SR14-1E (RR)×UGP 145 (rr) F₁ progenies SR14-76 A, B, C, D, and E were sib-mated. SR14-76C. # used to designate seedlings generated from seed harvested from mother plant SR14-76C. SR14-76C.11 was evaluated in and selected from the 2017 Seashore Paspalum Single Plant Nursery at UGA Griffin Campus and designated UGA 17-726.

### Example 5

### Performance Traits of UGA17-726

### Turf Quality and Performance

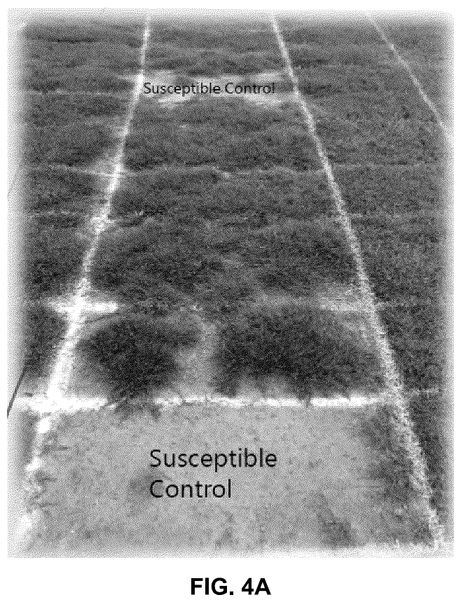

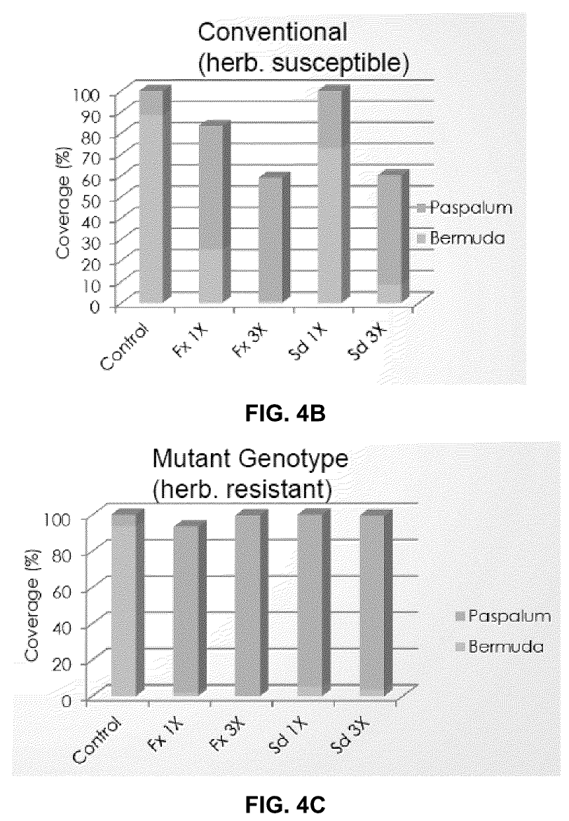

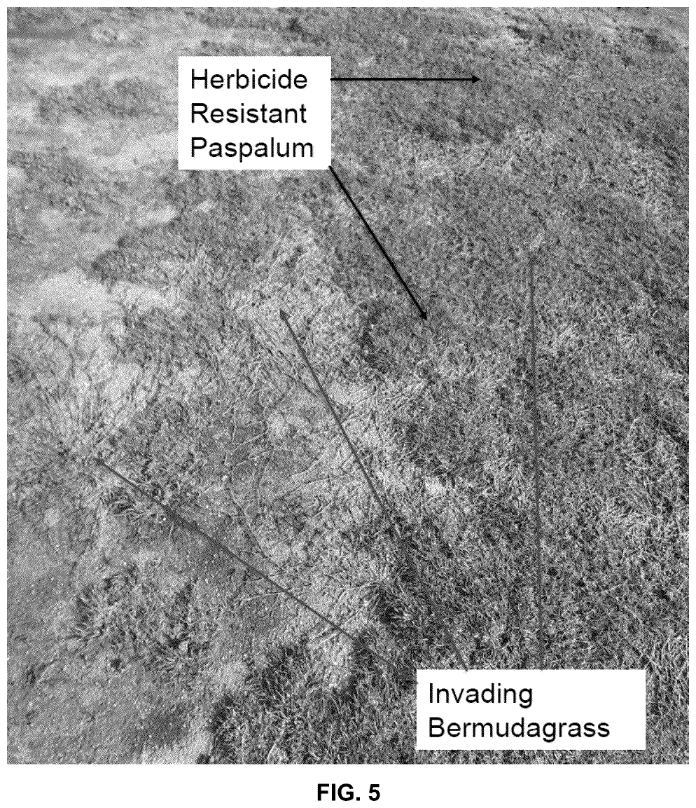

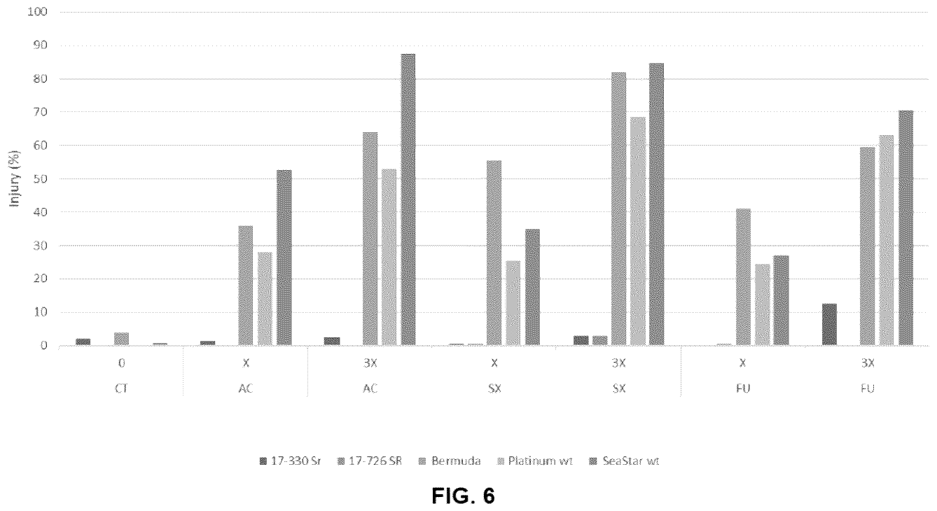

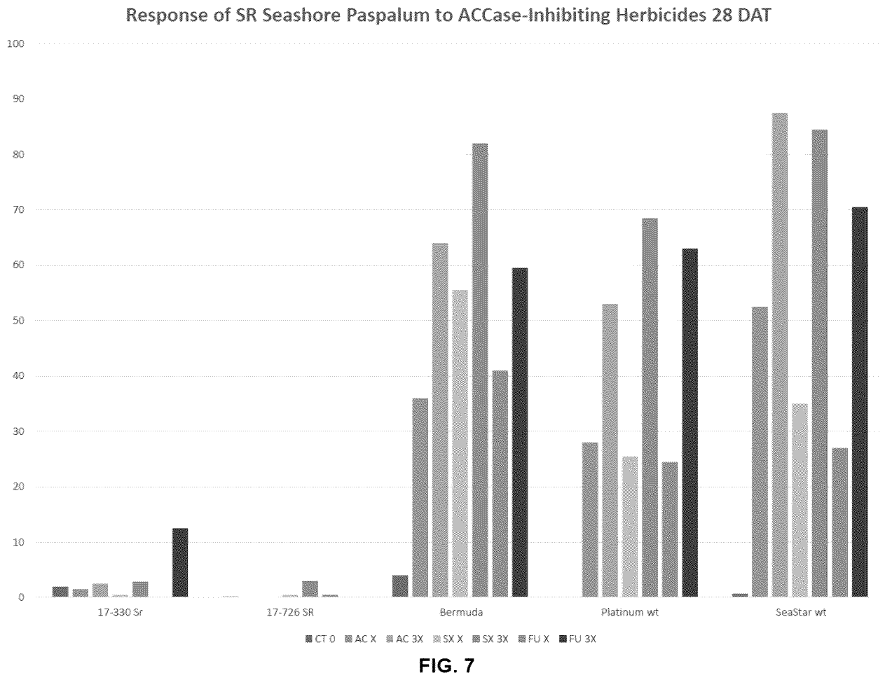

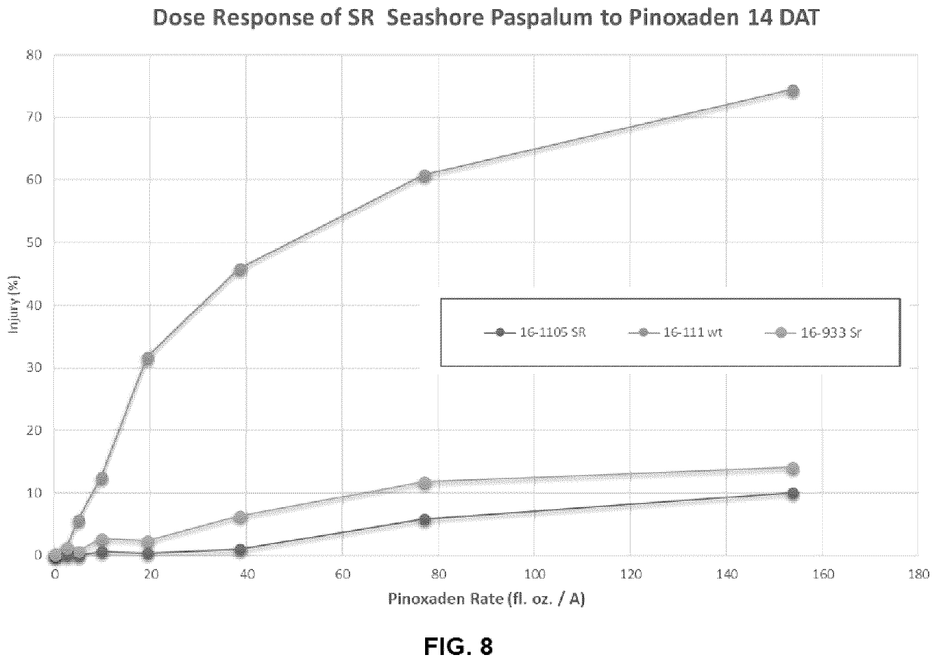

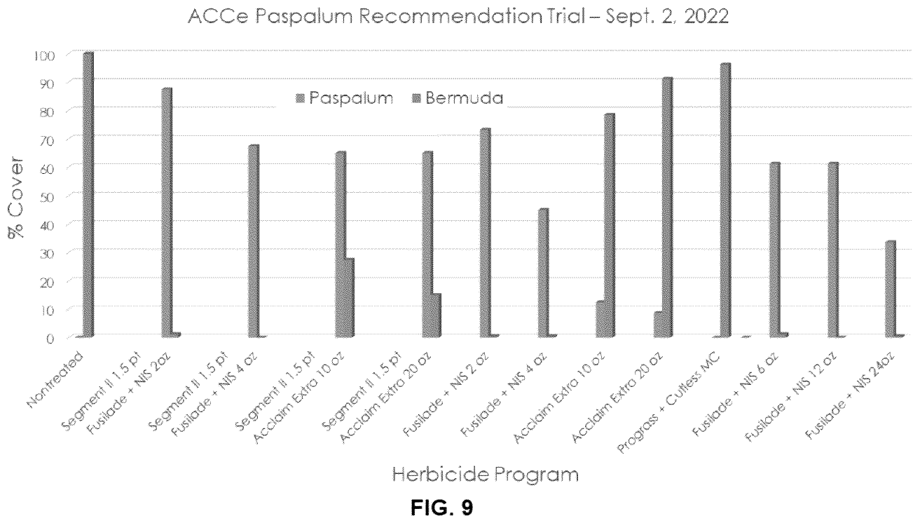

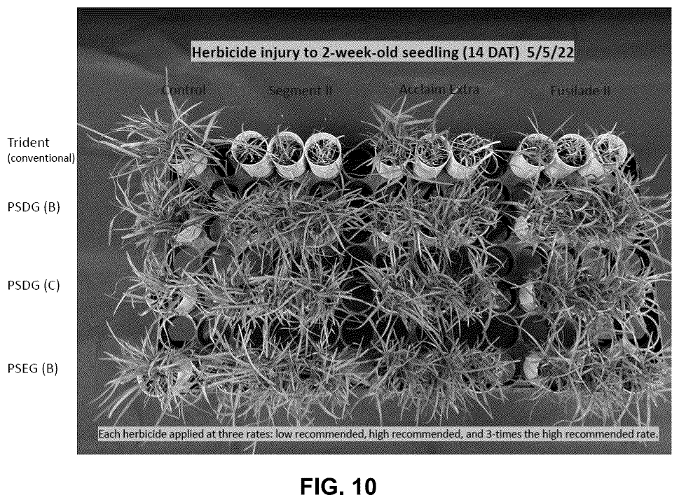

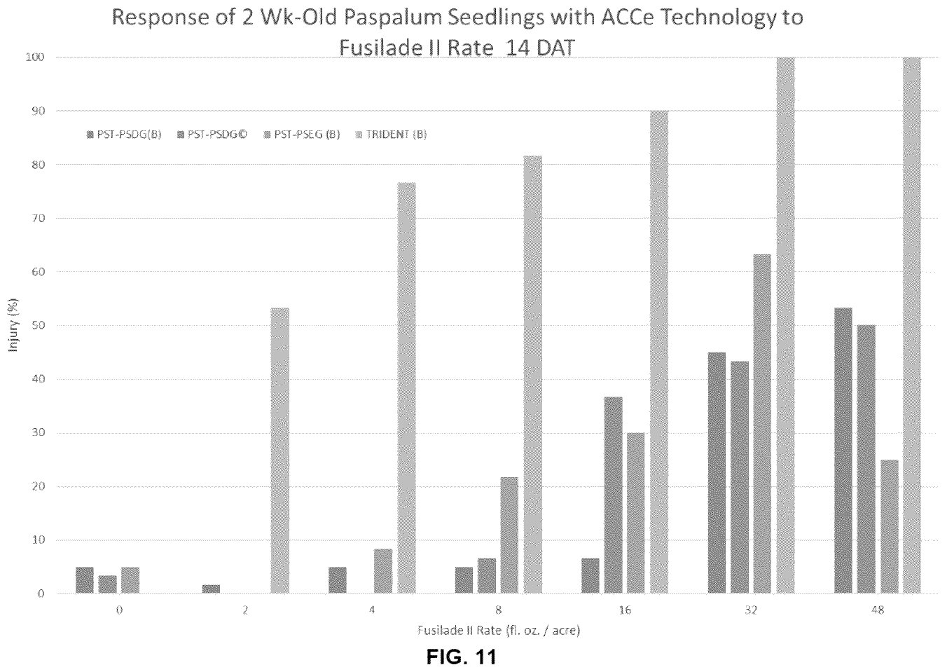

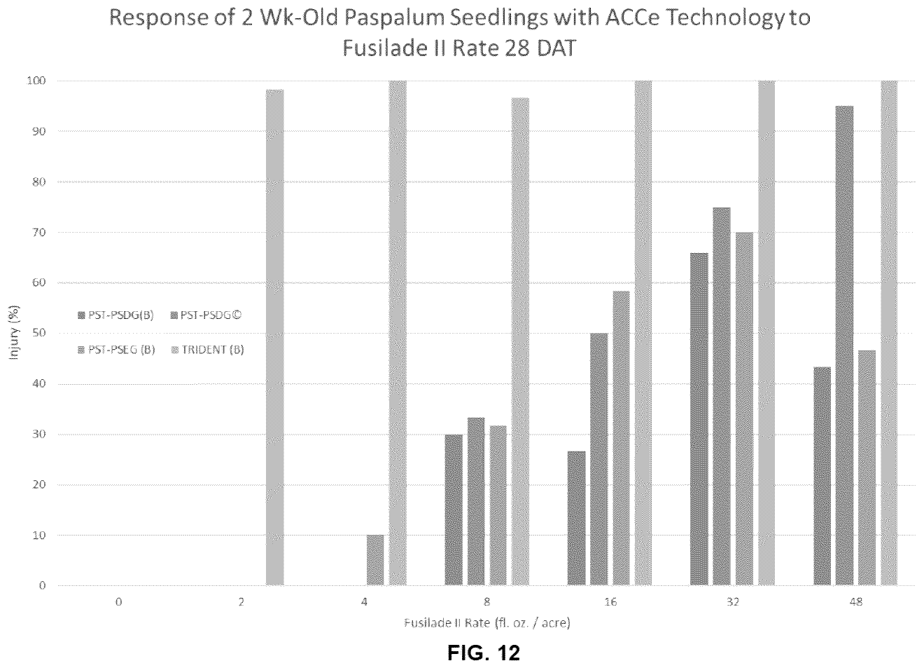

Field experiments were conducted using a control (no herbicide), clethodim at 1× and 3× (280 g/ha and 831 g/ha, respectively), fenoxyprop at 1× and 3× (191 g/ha and 372 g/ha, respectively), and sethoxydim at 1× and 3× (314 g/ha and 942 g/ha, respectively). Results can be seen in FIGS. 4A-4C.

TABLE 2
ADVANCED SEASHORE PASPALUM TRIAL - Planted 2020 - Griffin Campus

| | 8.12.21 | 7.1.22 | 7.1.22 | 8.18.22 | 8.18.22 | 8.18.22 |
|---|---|---|---|---|---|---|
| Line | Cover | Turf Quality | Dollar Spot | Quality | Color | NDVI |
| SeaScape | 91.7 | 6.7 | 1.0 | 7.6 | 8.7 | 0.77 |
| UGP145 | 76.7 | 6.8 | 1.3 | 7.7 | 7.9 | 0.74 |
| SeaStar | 90.0 | 6.0 | 15.0 | 7.1 | 7.9 | 0.71 |
| SeaDwarf | 86.7 | 6.1 | 3.0 | 6.8 | 7.6 | 0.70 |
| Platinum | 93.3 | 6.4 | 1.0 | 7.2 | 7.3 | 0.71 |
| 17-962 | 71.7 | 5.6 | 5.3 | 6.7 | 8.0 | 0.70 |
| 17-899 | 95.0 | 5.6 | 0.0 | 7.4 | 7.6 | 0.71 |
| 17-893 | 88.3 | 5.7 | 0.0 | 7.0 | 7.0 | 0.71 |
| 17-864 | 91.7 | 6.3 | 3.3 | 7.7 | 7.7 | 0.74 |
| 17-793 | 93.3 | 6.5 | 1.7 | 7.4 | 7.2 | 0.72 |
| 17-756 | 73.3 | 6.1 | 1.7 | 6.9 | 7.7 | 0.71 |
| 17-726 | 88.3 | 6.0 | 4.0 | 7.1 | 7.7 | 0.70 |
| 17-677 | 78.3 | 5.7 | 0.7 | 7.4 | 7.5 | 0.73 |
| 17-653 | 91.7 | 6.3 | 1.0 | 7.5 | 8.5 | 0.74 |
| 17-626 | 68.3 | 5.9 | 1.7 | 7.4 | 8.8 | 0.73 |
| 17-622 | 63.3 | 6.0 | 3.3 | 6.3 | 7.0 | 0.67 |
| 17-332 | 88.3 | 7.0 | 3.0 | 7.6 | 8.4 | 0.75 |
| 17-331 | 78.3 | 6.5 | 11.3 | 7.0 | 8.3 | 0.73 |
| 17-330 | 78.3 | 7.3 | 5.0 | 8.0 | 8.7 | 0.76 |
| 17-109 | 75.0 | 6.3 | 11.3 | 7.0 | 7.3 | 0.74 |
| 16-933 | 86.7 | 7.2 | 2.3 | 7.9 | 8.0 | 0.77 |
| 16-917 | 70.0 | 7.2 | 6.7 | 7.7 | 8.2 | 0.77 |
| 16-1609 | 78.3 | 6.3 | 12.3 | 7.0 | 8.2 | 0.72 |
| 16-1605 | 87.5 | 7.0 | 6.0 | 7.2 | 7.4 | 0.73 |
| 16-1571 | 83.3 | 5.6 | 1.7 | 7.5 | 8.0 | 0.74 |
| 16-1253 | 88.3 | 6.5 | 1.7 | 8.3 | 8.5 | 0.76 |
| 16-111 | 56.7 | 7.8 | 1.0 | 7.4 | 8.4 | 0.76 |
| 16-1022 | 53.3 | 5.8 | 18.3 | 7.0 | 8.0 | 0.74 |
| AVERAGE | 80.9 | 6.4 | 4.5 | 7.3 | 7.9 | 0.7 |

Note:
Bolded values are in the top statistical group according to L.S.D. at alpha = 0.05

### Example 6

### Morphological Traits and Description

Tables 3A-3B summarize morphological characteristics of UGA 17-726 in comparison with other turfgrass cultivars.

TABLE 3A
Comparison of Botanical Characteristics of Seashore Paspalum Cultivars

| | | | | UGP | UGA 17- |
|---|---|---|---|---|---|
| Platinum TE | SeaDwarf | SeaStar | UGA 17-330 | 73 | 726 |

Characteristics of Florets
Immature

| | | | | | | |
|---|---|---|---|---|---|---|
| Length | | | | | | |
| Obs. | | | | | | |
| 1 | 5.6 | 5.6 | 5.7 | 5.8 | 5 | 5.6 |
| 2 | 5.9 | 4.4 | 5.9 | 4.9 | 4.6 | 6 |
| 3 | 6 | 4.4 | 5.6 | 5.3 | 5.1 | 6.6 |
| Width | | | | | | |
| Obs. | | | | | | |
| 1 | 2.8 | 2.2 | 2.7 | 1.9 | 2.7 | 2.2 |
| 2 | 2.7 | 2.1 | 3 | 2.1 | 2.7 | 2.1 |
| 3 | 2.9 | 2.4 | 2.7 | 2.4 | 2.9 | 2.5 |
| Color | | | | | | |
| Obs. | | | | | | |
| 1 | 146C | 146D | 147C | 148C | 148C | 148D |
| 2 | 146C | 146C | 148D | 148C | 148D | 148C |
| 3 | 147C | 146D | 147C | 148D | 148C | 148B |
| Mature | | | | | | |
| Length | | | | | | |
| Obs. | | | | | | |
| 1 | 5.6 | 4.5 | 5.7 | 5.1 | 5.5 | 6.1 |
| 2 | 5.5 | 4.3 | 5.8 | 4.8 | 5.6 | 6 |
| 3 | 5 | 4.4 | 5.6 | 4.9 | 5.2 | 5.9 |
| Width | | | | | | |
| Obs. | | | | | | |
| 1 | 2 | 2.2 | 2.4 | 2.1 | 1.9 | 2.6 |
| 2 | 2.6 | 2.2 | 2.7 | 2 | 2.3 | 2.5 |
| 3 | 2.4 | 2.1 | 2.5 | 1.9 | 2.6 | 2.4 |
| Color | | | | | | |
| Obs. | | | | | | |
| 1 | 159A | 158A | 159A | 159B | 159B | 159B |
| 2 | 158A | 158A | 159B | 159B | 158A | 159B |
| 3 | 158A | 159A | 159A | 159A | 159B | 159B |

Characteristics of Seed

| | | | | | |
|---|---|---|---|---|---|
| Length | | | | | |
| Obs. | | | | | |
| 1 | 5.3 | 6 | 5.4 | 4.6 | 5.5 |
| 2 | 6.6 | 5.1 | 5.5 | | 6 |
| 3 | 5.9 | 5 | 4.9 | | 4.3 |
| Width | | | | | |
| Obs. | | | | | |
| 1 | 2.1 | 2.3 | 2.3 | 2.1 | 2.6 |
| 2 | 2.3 | 2.7 | 2.5 | | 2.4 |
| 3 | 2.4 | 2.4 | 2.4 | | 2.4 |
| Color | | | | | |
| Obs. | | | | | |
| 1 | 159A | 159B | 158A | 159A | 159A |
| 2 | 159B | 159B | 159B | | 159A |
| 3 | 158A | 159A | 159B | | 159B |

TABLE 3B
Comparison of botanical characteristics of seashore paspalum cultivars

| Platinum | | | | UGA | UGP | UGA 17- |
|---|---|---|---|---|---|---|
| TE | SeaDwarf | SeaStar | SeaIsle 1 | 17-330 | 73 | 726 |

Characteristics of Flowering Tillers
Length of Peduncle (mm)

| | | | | | | | |
|---|---|---|---|---|---|---|---|
| Obs. | | | | | | | |
| 1 | 10 | 10 | 5 | 5 | 5 | 6 | 5 |
| 2 | 6 | 8 | 6 | 6 | 1 | 8 | 1 |
| 3 | 2 | 6 | 6 | 8 | 1 | 2 | 2 |
| 4 | 1 | 5 | 7 | 15 | 1 | 10 | 1 |
| 5 | 1 | 6 | 9 | 10 | 5 | 18 | 1 |
| 6 | 1 | 8 | 1 | 12 | 4 | 14 | 3 |
| 7 | 5 | 5 | 1 | 7 | 1 | 13 | 1 |
| 8 | 3 | 9 | 4 | 12 | 1 | 1 | 3 |
| 9 | 3 | 5 | 1 | 10 | 2 | 13 | 2 |
| 10 | 1 | 6 | 2 | 10 | 1 | 11 | 2 |
| Mean | 3.3 | 6.8 | 4.2 | 9.5 | 2.2 | 9.6 | 2.1 |
| S.D | 2.8 | 1.7 | 2.7 | 2.9 | 1.7 | 5.1 | 1.2 |

Diameter of peduncle (mm)

| | | | | | | | |
|---|---|---|---|---|---|---|---|
| Obs. | | | | | | | |
| 1 | 0.58 | 0.57 | 0.45 | 0.58 | 0.66 | 0.83 | 0.73 |
| 2 | 0.44 | 0.51 | 0.57 | 0.72 | 0.53 | 0.67 | 0.5 |
| 3 | 0.42 | 0.66 | 0.67 | 0.68 | 0.41 | 0.71 | 0.44 |
| 4 | 0.5 | 0.5 | 0.48 | 0.65 | 0.45 | 0.73 | 0.55 |
| 5 | 0.54 | 0.35 | 0.56 | 0.49 | 0.48 | 0.63 | 0.52 |
| 6 | 0.55 | 0.44 | 0.4 | 0.56 | 0.55 | 0.52 | 0.68 |
| 7 | 0.47 | 0.54 | 0.4 | 0.66 | 0.4 | 0.48 | 0.43 |
| 8 | 0.68 | 0.55 | 0.49 | 0.62 | 0.49 | 0.74 | 0.54 |
| 9 | 0.42 | 0.4 | 0.5 | 0.52 | 0.41 | 0.59 | 0.42 |
| 10 | 0.71 | 0.42 | 0.48 | 0.53 | 0.5 | 0.68 | 0.35 |
| Mean | 0.5 | 0.5 | 0.5 | 0.6 | 0.5 | 0.6 | 0.5 |
| S.D | 0.1 | 0.1 | 0.1 | 0.1 | 0.1 | 0.1 | 0.1 |

Length of longest spike (mm)

| | | | | | | | |
|---|---|---|---|---|---|---|---|
| Obs. | | | | | | | |
| 1 | 22 | 22 | 15 | 35 | 19 | 24 | 28 |
| 2 | 23 | 26 | 21 | 30 | 12 | 25 | 33 |
| 3 | 28 | 19 | 20 | 24 | 14 | 17 | 30 |
| 4 | 27 | 19 | 18 | 29 | 18 | 25 | 29 |
| 5 | 23 | 18 | 24 | 20 | 14 | 35 | 30 |
| 6 | 29 | 11 | 19 | 26 | 17 | 22 | 31 |
| 7 | 16 | 12 | 20 | 19 | 18 | 20 | 25 |
| 8 | 30 | 14 | 25 | 24 | 13 | 21 | 20 |
| 9 | 26 | 13 | 30 | 29 | 15 | 25 | 28 |
| 10 | 28 | 10 | 23 | 29 | 12 | 23 | 23 |
| Mean | 25.2 | 16.4 | 21.5 | 26.5 | 15.2 | 23.7 | 27.7 |
| S.D | 4 | 5 | 4 | 4.6 | 2.5 | 4.5 | 3.7 |

Number of spikes per inflorescence

| | | | | | | | |
|---|---|---|---|---|---|---|---|
| Obs. | | | | | | | |
| 1 | 2 | 2 | 2 | 2 | 2 | 2 | 2 |
| 2 | 2 | 2 | 2 | 2 | 2 | 2 | 2 |
| 3 | 2 | 2 | 2 | 2 | 2 | 2 | 2 |
| 4 | 2 | 2 | 2 | 2 | 2 | 2 | 2 |
| 5 | 2 | 2 | 2 | 2 | 2 | 2 | 2 |
| 6 | 2 | 2 | 2 | 2 | 2 | 2 | 2 |
| 7 | 2 | 2 | 2 | 2 | 2 | 2 | 2 |
| 8 | 2 | 2 | 2 | 2 | 2 | 2 | 2 |
| 9 | 2 | 2 | 2 | 2 | 2 | 2 | 2 |
| 10 | 2 | 2 | 2 | 2 | 2 | 2 | 2 |
| Mean | 2 | 2 | 2 | 2 | 2 | 2 | 2 |
| S.D | 0 | 0 | 0 | 0 | 0 | 0 | 0 |

Maximum number of spikes per inflorescence

| | | | | | | | |
|---|---|---|---|---|---|---|---|
| Obs. | | | | | | | |
| 1 | 2 | 2 | 2 | 2 | 2 | 2 | 2 |
| 2 | 2 | 2 | 2 | 2 | 2 | 2 | 2 |
| 3 | 2 | 2 | 2 | 2 | 2 | 2 | 2 |
| 4 | 2 | 2 | 2 | 2 | 2 | 2 | 2 |
| 5 | 2 | 2 | 2 | 2 | 2 | 2 | 2 |
| 6 | 2 | 2 | 2 | 2 | 2 | 2 | 2 |
| 7 | 2 | 2 | 2 | 2 | 2 | 2 | 2 |
| 8 | 2 | 2 | 2 | 2 | 2 | 2 | 2 |
| 9 | 2 | 2 | 2 | 2 | 2 | 2 | 2 |
| 10 | 2 | 2 | 2 | 2 | 2 | 2 | 2 |
| Mean | 2 | 2 | 2 | 2 | 2 | 2 | 2 |
| S.D | 0 | 0 | 0 | 0 | 0 | 0 | 0 |

Length of spike branch from flag leaf (mm)

| | | | | | | | |
|---|---|---|---|---|---|---|---|
| Obs. | | | | | | | |
| 1 | 7 | 11 | 3 | 4 | 4 | 9 | 10 |
| 2 | 9 | 9 | 5 | 3 | 1 | 5 | 14 |
| 3 | 6 | 7 | 5 | 5 | 1 | 1 | 8 |
| 4 | 8 | 3 | 6 | 6 | 1 | 7 | 13 |
| 5 | 9 | 5 | 10 | 13 | 6 | 16 | 5 |
| 6 | 8 | 6 | 1 | 12 | 4 | 12 | 6 |
| 7 | 12 | 4 | 4 | 11 | 5 | 14 | 12 |
| 8 | 11 | 7 | 8 | 12 | 4 | 1 | 8 |
| 9 | 5 | 6 | 4 | 4 | 3 | 15 | 13 |
| 10 | 11 | 4 | 7 | 8 | 6 | 9 | 9 |
| Mean | 8.6 | 6.2 | 5.3 | 7.8 | 3.5 | 8.9 | 9.8 |
| S.D | 2.2 | 2.3 | 2.5 | 3.7 | 1.9 | 5.20 | 3 |

Florets per spike

| | | | | | | | |
|---|---|---|---|---|---|---|---|
| Obs. | | | | | | | |
| 1 | 16 | 17 | 15 | 23 | 16 | 23 | 21 |
| 2 | 16 | 24 | 19 | 21 | 11 | 16 | 23 |
| 3 | 16 | 16 | 21 | 18 | 11 | 14 | 21 |
| 4 | 16 | 18 | 17 | 20 | 17 | 17 | 21 |
| 5 | 16 | 16 | 17 | 14 | 12 | 10 | 22 |
| 6 | 21 | 9 | 18 | 15 | 15 | 17 | 23 |
| 7 | 9 | 9 | 20 | 21 | 12 | 13 | 17 |
| 8 | 20 | 16 | 23 | 19 | 10 | 15 | 12 |
| 9 | 15 | 15 | 23 | 26 | 18 | 17 | 19 |
| 10 | 17 | 10 | 20 | 22 | 16 | 15 | 15 |
| Mean | 16.2 | 15 | 19.3 | 19.9 | 13.8 | 15.7 | 19.4 |
| S.D | 3 | 4 | 2.5 | 3.4 | 2.8 | 3.2 | 3.5 |

Length of blade on flag leaf (mm)

| | | | | | | | |
|---|---|---|---|---|---|---|---|
| Obs. | | | | | | | |
| 1 | 14 | 8 | 9 | 14 | 15 | 6 | 26 |
| 2 | 23 | 5 | 11 | 6 | 16 | 6 | 12 |
| 3 | 20 | 10 | 13 | 10 | 16 | 8 | 25 |
| 4 | 23 | 11 | 6 | 11 | 14 | 7 | 9 |
| 5 | 23 | 9 | 5 | 10 | 19 | 3 | 22 |
| 6 | 10 | 9 | 9 | 15 | 24 | 4 | 13 |
| 7 | 12 | 8 | 11 | 10 | 8 | 10 | 25 |
| 8 | 25 | 13 | 7 | 15 | 3 | 5 | 6 |
| 9 | 18 | 8 | 11 | 5 | 7 | 7 | 16 |
| 10 | 21 | 10 | 8 | 6 | 6 | 4 | 25 |
| Mean | 18.9 | 9.1 | 9 | 10.2 | 12.8 | 6 | 17.9 |
| S.D | 4.9 | 2 | 2.4 | 3.5 | 6.2 | 2 | 7.2 |

Width of blade on flat leaf (mm)

| | | | | | | | |
|---|---|---|---|---|---|---|---|
| Obs. | | | | | | | |
| 1 | 0.6 | 0.3 | 0.8 | 1 | 1 | 0.8 | 1 |
| 2 | 0.7 | 0.25 | 0.9 | 0.4 | 1.5 | 1 | 1 |
| 3 | 1 | 1 | 0.6 | 0.9 | 0.6 | 1 | 0.6 |
| 4 | 0.75 | 1 | 0.5 | 0.75 | 0.5 | 1.2 | 1 |
| 5 | 0.7 | 0.9 | 0.4 | 0.35 | 0.5 | 1 | 0.65 |
| 6 | 0.5 | 0.2 | 0.7 | 0.4 | 0.9 | 0.9 | 0.8 |
| 7 | 0.5 | 0.5 | 1 | 1 | 0.5 | 1 | 0.7 |
| 8 | 1 | 1 | 1 | 0.5 | 0.5 | 0.6 | 1 |
| 9 | 0.8 | 1 | 0.9 | 0.35 | 1 | 0.7 | 0.6 |
| 10 | 1 | 0.9 | 0.8 | 0.4 | 0.5 | 0.7 | 1 |
| Mean | 0.8 | 0.7 | 0.8 | 0.6 | 0.8 | 0.9 | 0.8 |
| S.D | 0.2 | 0.3 | 0.2 | 0.3 | 0.3 | 0.2 | 0.2 |

Length of sheath on flag leaf (mm)

| | | | | | | | |
|---|---|---|---|---|---|---|---|
| Obs. | | | | | | | |
| 1 | 41 | 33 | 28 | 55 | 25 | 32 | 42 |
| 2 | 43 | 42 | 36 | 42 | 25 | 33 | 46 |
| 3 | 48 | 27 | 32 | 40 | 26 | 28 | 37 |
| 4 | 49 | 27 | 35 | 35 | 27 | 32 | 36 |
| 5 | 41 | 19 | 18 | 53 | 20 | 44 | 45 |
| 6 | 43 | 22 | 25 | 30 | 22 | 31 | 47 |
| 7 | 36 | 23 | 35 | 43 | 20 | 31 | 58 |
| 8 | 57 | 32 | 38 | 54 | 19 | 32 | 39 |
| 9 | 49 | 20 | 29 | 43 | 22 | 38 | 45 |
| 10 | 45 | 22 | 20 | 50 | 20 | 32 | 43 |
| Mean | 45.2 | 26.7 | 29.6 | 44.5 | 22.6 | 33.3 | 43.8 |
| S.D | 5.5 | 6.8 | 6.5 | 8 | 2.8 | 4.3 | 6 |

Length of blade on 4th leaf (mm)

| | | | | | | | |
|---|---|---|---|---|---|---|---|
| Obs. | | | | | | | |
| 1 | 45 | 34 | 48 | 66 | 46 | 40 | 67 |
| 2 | 45 | 44 | 53 | 53 | 46 | 39 | 61 |
| 3 | 46 | 30 | 55 | 66 | 40 | 35 | 70 |
| 4 | 50 | 43 | 29 | 58 | 56 | 56 | 47 |
| 5 | 50 | 34 | 40 | 33 | 60 | 60 | 58 |
| 6 | 54 | 29 | 42 | 40 | 47 | 35 | 70 |
| 7 | 41 | 37 | 49 | 59 | 31 | 29 | 55 |
| 8 | 47 | 51 | 41 | 76 | 30 | 31 | 48 |
| 9 | 55 | 40 | 38 | 54 | 30 | 24 | 41 |
| 10 | 49 | 39 | 35 | 55 | 45 | 25 | 73 |
| Mean | 48.2 | 38.1 | 43 | 56 | 43.1 | 37.4 | 59 |
| S.D | 4.1 | 6.4 | 7.8 | 11.9 | 9.9 | 11.5 | 10.5 |

Width of blade on 4th leaf (mm)

| | | | | | | | |
|---|---|---|---|---|---|---|---|
| Obs. | | | | | | | |
| 1 | 2 | 1 | 3 | 3 | 2 | 3.5 | 3 |
| 2 | 3 | 3 | 3 | 3 | 3.5 | 3.5 | 3 |
| 3 | 3 | 1.5 | 3 | 3.5 | 2.5 | 3 | 4 |
| 4 | 3 | 2.5 | 2.5 | 3 | 3 | 3 | 3 |
| 5 | 3 | 1.5 | 3.5 | 2.5 | 2 | 3.5 | 2 |
| 6 | 4 | 3 | 3 | 3 | 2 | 3 | 1 |
| 7 | 3 | 3 | 3.5 | 3.5 | 2 | 3 | 3 |
| 8 | 3 | 2 | 4 | 2.5 | 3.5 | 4 | 2 |
| 9 | 4 | 2 | 3.5 | 3 | 2.5 | 3 | 2 |
| 10 | 3 | 2 | 2.5 | 3 | 2.5 | 1.5 | 4 |
| Mean | 3.1 | 2.2 | 3.2 | 3 | 2.6 | 3.1 | 2.7 |
| S.D | 0.5 | 0.7 | 0.5 | 0.3 | 0.6 | 0.6 | 0.9 |

Length of sheath on 4th leaf (mm)

| | | | | | | | |
|---|---|---|---|---|---|---|---|
| Obs. | | | | | | | |
| 1 | 16 | 13 | 14 | 21 | 13 | 12 | 20 |
| 2 | 12 | 17 | 17 | 20 | 12 | 13 | 19 |
| 3 | 16 | 15 | 18 | 20 | 15 | 12 | 23 |
| 4 | 14 | 14 | 16 | 17 | 9 | 20 | 20 |
| 5 | 10 | 11 | 15 | 14 | 12 | 18 | 7 |
| 6 | 15 | 7 | 17 | 15 | 10 | 13 | 11 |
| 7 | 14 | 6 | 16 | 16 | 18 | 14 | 18 |
| 8 | 16 | 16 | 19 | 13 | 13 | 13 | 17 |
| 9 | 17 | 14 | 14 | 17 | 10 | 14 | 15 |
| 10 | 16 | 10 | 15 | 16 | 11 | 12 | 16 |
| Mean | 14.6 | 12.3 | 16.1 | 16.9 | 12.3 | 14.1 | 16.6 |
| S.D | 2.1 | 3.5 | 1.6 | 2.5 | 2.5 | 2.6 | 4.5 |

Length of 4th internode (mm)

| | | | | | | | |
|---|---|---|---|---|---|---|---|
| Obs. | | | | | | | |
| 1 | 13 | 5 | 6 | 14 | 10 | 17 | 20 |
| 2 | 10 | 6 | 5 | 8 | 22 | 10 | 19 |
| 3 | 16 | 7 | 12 | 13 | 20 | 11 | 23 |
| 4 | 11 | 6 | 11 | 8 | 19 | 13 | 20 |
| 5 | 7 | 5 | 8 | 9 | 7 | 13 | 7 |
| 6 | 8 | 3 | 8 | 9 | 10 | 7 | 11 |
| 7 | 11 | 6 | 7 | 14 | 7 | 10 | 18 |
| 8 | 9 | 8 | 8 | 15 | 5 | 8 | 17 |
| 9 | 15 | 4 | 6 | 9 | 5 | 19 | 15 |
| 10 | 16 | 3 | 7 | 14 | 8 | 5 | 16 |
| Mean | 11.6 | 5.3 | 7.8 | 11.3 | 11.4 | 11.3 | 16.6 |
| S.D | 3.1 | 1.6 | 2.1 | 2.8 | 6.2 | 4.1 | 4.5 |

Characteristics of Stolons
Length of 4th internode (mm)

| | | | | | | | |
|---|---|---|---|---|---|---|---|
| Obs. | | | | | | | |
| 1 | 9 | 12 | 11 | 19 | 17 | 17 | 15 |
| 2 | 22 | 11 | 14 | 9 | 17 | 12 | 18 |
| 3 | 16 | 15 | 9 | 16 | 16 | 16 | 10 |
| 4 | 25 | 10 | 12 | 15 | 13 | 7 | 7 |
| 5 | 19 | 10 | 13 | 11 | 15 | 11 | 20 |
| 6 | 10 | 15 | 13 | 15 | 12 | 13 | 15 |
| 7 | 18 | 8 | 10 | 19 | 10 | 12 | 16 |
| 8 | 20 | 12 | 10 | 10 | 10 | 10 | 15 |
| 9 | 18 | 15 | 12 | 14 | 12 | 21 | 11 |
| 10 | 21 | 13 | 11 | 12 | 11 | 19 | 10 |
| Mean | 17.8 | 12.1 | 11.5 | 14 | 13.3 | 13.8 | 13.7 |
| S.D | 4.8 | 2.3 | 1.5 | 3.3 | 2.6 | 4.1 | 3.8 |

Diameter of 4th internode (mm)

| | | | | | | | |
|---|---|---|---|---|---|---|---|
| Obs. | | | | | | | |
| 1 | 1.91 | 1.4 | 1.25 | 1.76 | 1.34 | 1.88 | 2.01 |
| 2 | 2.24 | 1.54 | 1.3 | 1.79 | 1.42 | 1.53 | 1.61 |
| 3 | 1.88 | 1.65 | 1.77 | 1.48 | 1.47 | 1.67 | 1.69 |
| 4 | 2.4 | 1.21 | 1.4 | 1.44 | 1.33 | 1.81 | 1.49 |
| 5 | 2.15 | 1.23 | 1.33 | 1.3 | 1.5 | 1.92 | 1.76 |
| 6 | 2.27 | 1.55 | 1.47 | 1.67 | 1.43 | 1.55 | 1.41 |
| 7 | 2.13 | 1.66 | 1.46 | 1.8 | 1.24 | 1.53 | 1.52 |
| 8 | 1.78 | 1.49 | 1.58 | 1.4 | 1.37 | 1.53 | 1.62 |
| 9 | 1.94 | 1.31 | 1.62 | 1.45 | 1.41 | 1.41 | 1.41 |
| 10 | 1.96 | 1.22 | 1.43 | 1.49 | 1.39 | 1.4 | 1.65 |
| Mean | 2.1 | 1.4 | 1.5 | 1.6 | 1.4 | 1.6 | 1.6 |
| S.D | 0.2 | 0.2 | 0.2 | 0.2 | 0.1 | 0.2 | 0.2 |

Length of 4th leaf blade (mm)

| | | | | | | | |
|---|---|---|---|---|---|---|---|
| Obs. | | | | | | | |
| 1 | 7 | 4 | 5 | 8 | 3 | 3 | 15 |
| 2 | 9 | 3 | 4 | 7 | 4 | 3 | 4 |
| 3 | 5 | 4 | 4 | 6 | 2 | 4 | 4 |
| 4 | 7 | 6 | 5 | 17 | 2 | 7 | 5 |
| 5 | 5 | 3 | 5 | 8 | 3 | 6 | 5 |
| 6 | 4 | 4 | 6 | 9 | 2 | 14 | 10 |
| 7 | 6 | 5 | 5 | 5 | 4 | 9 | 12 |
| 8 | 6 | 6 | 4 | 15 | 3 | 12 | 15 |
| 9 | 7 | 6 | 5 | 9 | 3 | 3 | 14 |
| 10 | 10 | 2 | 4 | 8 | 3 | 6 | 8 |
| Mean | 6.6 | 4.3 | 4.7 | 9.2 | 2.9 | 6.7 | 9.2 |
| S.D | 1.7 | 1.3 | 0.6 | 3.6 | 0.7 | 3.7 | 4.4 |

Width of 4th leaf blade (mm)

| | | | | | | | |
|---|---|---|---|---|---|---|---|
| Obs. | | | | | | | |
| 1 | 3 | 1 | 1.5 | 2 | 1.5 | 1.5 | 3 |
| 2 | 2 | 1.5 | 1 | 2 | 1 | 1.5 | 1.5 |
| 3 | 3 | 2.5 | 1.5 | 2.5 | 1 | 1.5 | 1 |
| 4 | 2.5 | 1 | 2 | 3 | 0.5 | 3 | 1 |
| 5 | 2 | 1 | 1.5 | 1 | 1 | 3 | 2 |
| 6 | 2 | 1 | 2 | 1.5 | 0.5 | 3.5 | 1.5 |
| 7 | 2.5 | 1 | 1 | 3 | 1 | 3 | 3 |
| 8 | 2 | 2 | 1.5 | 3 | 1 | 3 | 3 |
| 9 | 3 | 1 | 2 | 2 | 1 | 2.5 | 2 |
| 10 | 4 | 1 | 1.5 | 1 | 1 | 3 | 3 |
| Mean | 2.6 | 1.3 | 1.6 | 2.1 | 1 | 2.6 | 2.1 |
| S.D | 0.6 | 0.5 | 0.4 | 0.7 | 0.3 | 0.7 | 0.8 |

Length of leaf sheath on 4th node (mm)

| | | | | | | | |
|---|---|---|---|---|---|---|---|
| Obs. | | | | | | | |
| 1 | 6 | 5 | 7 | 9 | 7 | 8 | 10 |
| 2 | 10 | 5 | 6 | 7 | 6 | 9 | 8 |
| 3 | 8 | 4 | 6 | 8 | 6 | 8 | 6 |
| 4 | 8 | 4 | 7 | 13 | 5 | 7 | 6 |
| 5 | 9 | 6 | 6 | 8 | 6 | 7 | 10 |
| 6 | 7 | 5 | 7 | 7 | 5 | 9 | 9 |
| 7 | 9 | 4 | 6 | 8 | 4 | 10 | 9 |
| 8 | 8 | 4 | 6 | 12 | 5 | 9 | 12 |
| 9 | 8 | 7 | 8 | 8 | 6 | 10 | 12 |
| 10 | 10 | 4 | 7 | 9 | 5 | 9 | 10 |
| Mean | 8.3 | 4.8 | 6.6 | 8.9 | 5.5 | 8.6 | 9.2 |
| S.D | 1.2 | 1 | 0.7 | 1.9 | 0.8 | 1 | 2 |

Other Characteristics
Culm height (cm)

| | | | | | | | |
|---|---|---|---|---|---|---|---|
| Obs. | | | | | | | |
| 1 | 22 | 4 | 10 | 29 | 10 | 14 | 13 |
| 2 | 12 | 6 | 11 | 20 | 8 | 13 | 20 |
| 3 | 15 | 3 | 13 | 18 | 9 | 15 | 19 |
| 4 | 18 | 5 | 12 | 25 | 7 | 15 | 21 |
| 5 | 19 | 7 | 14 | 20 | 6 | 16 | 16 |
| 6 | 20 | 8 | 9 | 24 | 8 | 18 | 18 |
| 7 | 22 | 3 | 10 | 23 | 8 | 17 | 20 |
| 8 | 20 | 4 | 11 | 18 | 10 | 19 | 20 |
| 9 | 18 | 8 | 12 | 21 | 9 | 19 | 18 |
| 10 | 19 | 7 | 12 | 25 | 8 | 18 | 17 |
| Mean | 18.5 | 5.5 | 11.4 | 22.3 | 8.3 | 16.4 | 18.2 |
| S.D | 2.9 | 1.9 | 1.4 | 3.3 | 1.2 | 2 | 2.3 |

Seed head height (cm)

| | | | | | | | |
|---|---|---|---|---|---|---|---|
| Obs. | | | | | | | |
| 1 | 15 | 3 | 7.5 | 22 | 5.5 | 5 | 12 |
| 2 | 21 | 2 | 6 | 18 | 6 | 8 | 18 |
| 3 | 19 | 2.5 | 8 | 17 | 3 | 6 | 11 |
| 4 | 18 | 4 | 7 | 19 | 4 | 5 | 19 |
| 5 | 20 | 3 | 7.5 | 13 | 4.5 | 4 | 21 |
| 6 | 13 | 3 | 6.5 | 18 | 3 | 6 | 17 |
| 7 | 19 | 2 | 8 | 17 | 3.5 | 7 | 15 |
| 8 | 17 | 5 | 7.5 | 21 | 5 | 2 | 13 |
| 9 | 16 | 2 | 6 | 14 | 5 | 3 | 15 |
| 10 | 19 | 2.5 | 5 | 15 | 6 | 2 | 12 |
| Mean | 17.7 | 2.9 | 6.9 | 17.4 | 4.6 | 4.8 | 15.3 |
| S.D | 2.3 | 0.9 | 0.9 | 2.7 | 1.1 | 1.9 | 3.2 |

Color of upper leaf surface

| | | | | | | |
|---|---|---|---|---|---|---|
| 138 B | 137 D | 137 D | 137 D | 137 A | 137 B | 137 C |

Color of lower leaf surface

| | | | | | | |
|---|---|---|---|---|---|---|
| 137 A | 137 C | 137 C | 137 C | 137 B | 137 C | 137 B |

Stolon Color

| | | | | | | |
|---|---|---|---|---|---|---|
| 146 C | 146 B | 146 D | 146 D | 146 B | 146 D | 146 B |

Anther Color

| | | | | | | |
|---|---|---|---|---|---|---|
| 83 B | 83 B | 83 A | 83 A | 83 A | 83 A | 83 B |

Stigma Color

| | | | | | | |
|---|---|---|---|---|---|---|
| 86 A | 86 A | 86 B | 86 B | 86 A | 86 A | 86 A |

### Example 7

### Performance of Seeded Cultivars Using 17-726 as a Parent

G21SSP_Seeded Plm data summary provides evidence of performance of seeded cultivar PSDG (17-726 is one of three parental lines used to produce the cultivar).

TABLE 4
2019 Seeded Seashore Paspalum Preliminary Trial - Griffin, GA

| | | Initial | Qual | Stand | Qual | Greenup | Dollar | Qual | Avg |
|---|---|---|---|---|---|---|---|---|---|
| | | Stand | ′21 | ′21 | ′22 | ′23 | Spot | ′23 | Qual |
| Line | Type | (1-9) | (1-9) | % | (1-9) | % | % | (1-9) | (1-9) |
| SEASPRAY | Seeded | | 5.4 | 65.0 | 4.5 | 20.0 | 15.0 | 4.2 | 4.7 |
| Sr16-1609 | Vegetative | | 4.8 | 52.5 | 5.2 | 1.7 | 10.0 | 4.6 | 4.9 |
| SEASTAR | Vegetative | | 6.3 | 78.3 | 5.6 | 31.7 | 16.7 | 5.0 | 5.6 |
| PURE | Seeded | 8.1 | 5.8 | 98.8 | 6.4 | 43.8 | 11.3 | 5.5 | 6.0 |
| DYNASTY | | | | | | | | | |
| PST-PEY | Seeded | 9.0 | 5.7 | 100.0 | 5.8 | 10.7 | 13.3 | 5.8 | 5.8 |
| PST-PGA | Seeded | 6.7 | 5.7 | 97.7 | 5.5 | 20.0 | 16.7 | 5.8 | 5.6 |
| PST-PSDG | Seeded | 5.7 | 6.5 | 99.3 | 6.5 | 31.7 | 15.0 | 5.8 | 6.3 |
| PST-PS3 | Seeded | 7.3 | 5.4 | 98.3 | 6.6 | 26.7 | 14.0 | 5.9 | 6.0 |
| TRITON | Seeded | 7.3 | 5.6 | 99.3 | 6.4 | 45.0 | 16.7 | 6.0 | 6.0 |
| PLATINUM | Vegetative | | 5.2 | 63.3 | 5.8 | 5.0 | 7.3 | 6.0 | 5.7 |
| HYB NEW | Seeded | 2.0 | 6.4 | 88.3 | 6.0 | 33.3 | 16.3 | 6.0 | 6.2 |
| NEPTUNE | Seeded | 8.3 | 6.3 | 100.0 | 6.2 | 45.0 | 21.7 | 6.2 | 6.2 |
| PST-SS2 | Seeded | 7.3 | 5.8 | 99.3 | 6.4 | 35.0 | 20.0 | 6.3 | 6.2 |
| HYB 2 | Seeded | 1.0 | 6.3 | 75.0 | 5.9 | 26.7 | 25.0 | 6.3 | 6.1 |
| SYN-PIX | Seeded | 8.8 | 5.9 | 100.0 | 5.8 | 23.3 | 21.7 | 6.4 | 6.1 |
| TRIDENT | Seeded | 8.7 | 5.6 | 100.0 | 6.4 | 48.3 | 15.0 | 6.7 | 6.2 |
| Average | | 7.1 | 6.0 | 99.0 | 5.9 | 20.8 | 15.0 | 5.8 | 5.9 |

Notes:
PST-PSDG is produced using UGA 17-726 as one of three parental lines.
Bolded values are in the top statistical group according to Student's t-test.
Entries highlighted in yellow were established vegetatively.

### Example 10

### 17-726 Herbicide Tolerance Experiments

ACCase herbicides were applied Oct. 11, 2023. 14 day injury was assessed on Oct. 25, 2023. 28 day injury was assessed on Nov. 8, 2023. SX=sethoxydim; FU=fluazifop-P; and AC=fenoxaprop-P.

TABLE 5
Herbicide Tolerance Experiments

| | | | | Injury | Injury | Injury | | | Injury | | |
|---|---|---|---|---|---|---|---|---|---|---|---|
| Cultivar | Herb | Rate | Rep | (%) | (%) | 14 d | Injury (%) | | 28 d | f.w. | d.w. |
| SeaStar | SX | X | 1 | 20 | 20 | 20 | 20 | 50 | 35 | 1.818 | 0.673 |
| SeaStar | SX | 3X | 1 | 70 | 70 | 70 | 80 | 90 | 85 | 1.597 | 0.978 |
| SeaStar | FU | X | 1 | 5 | 15 | 10 | 5 | 20 | 12.5 | 3.188 | 0.913 |
| SeaStar | FU | 3X | 1 | 50 | 30 | 40 | 60 | 70 | 65 | 2.313 | 1.208 |
| SeaStar | AC | X | 1 | 40 | 20 | 30 | 20 | 40 | 30 | 2.676 | 0.968 |
| SeaStar | AC | 3X | 1 | 60 | 35 | 47.5 | 70 | 80 | 75 | 1.381 | 0.833 |
| SeaStar | CT | 0 | 1 | 0 | 0 | 0 | 0 | 0 | 0 | 11.947 | 2.482 |
| Platinum | SX | X | 1 | 10 | 15 | 12.5 | 10 | 30 | 20 | 3.401 | 1.224 |
| Platinum | SX | 3X | 1 | 60 | 40 | 50 | 60 | 90 | 75 | 1.148 | 0.655 |
| Platinum | FU | X | 1 | 15 | 15 | 15 | 15 | 40 | 27.5 | 3.954 | 1.482 |
| Platinum | FU | 3X | 1 | 50 | 25 | 37.5 | 85 | 80 | 82.5 | 1.703 | 1.075 |
| Platinum | AC | X | 1 | 40 | 35 | 37.5 | 25 | 60 | 42.5 | 3.249 | 1.229 |
| Platinum | AC | 3X | 1 | 35 | 35 | 35 | 50 | 70 | 60 | 2.462 | 1.263 |
| Platinum | CT | 0 | 1 | 0 | 0 | 0 | 0 | 0 | 0 | 10.006 | 2.224 |
| 17-726 | SX | X | 1 | 0 | 0 | 0 | 0 | 0 | 0 | 8.911 | 1.737 |
| 17-726 | SX | 3X | 1 | 0 | 0 | 0 | 0 | 0 | 0 | 8.094 | 1.699 |
| 17-726 | FU | X | 1 | 5 | 5 | 5 | 5 | 0 | 2.5 | 10.771 | 2.326 |
| 17-726 | FU | 3X | 1 | 0 | 0 | 0 | 0 | 0 | 0 | 4.012 | 0.855 |
| 17-726 | AC | X | 1 | 0 | 0 | 0 | 0 | 0 | 0 | 9.152 | 1.74 |
| 17-726 | AC | 3X | 1 | 0 | 0 | 0 | 0 | 0 | 0 | 9.347 | 1.846 |
| 17-726 | CT | 0 | 1 | 0 | 0 | 0 | 0 | 2 | 1 | 11.013 | 2.576 |
| 17-330 | SX | X | 1 | 0 | 0 | 0 | 0 | 0 | 0 | 7.913 | 1.342 |
| 17-330 | SX | 3X | 1 | 5 | 5 | 5 | 2 | 5 | 3.5 | 6.473 | 1.133 |
| 17-330 | FU | X | 1 | 0 | 0 | 0 | 0 | 0 | 0 | 6.933 | 1.401 |
| 17-330 | FU | 3X | 1 | 10 | 10 | 10 | 10 | 20 | 15 | 4.424 | 1.194 |
| 17-330 | AC | X | 1 | 0 | 0 | 0 | 0 | 0 | 0 | 4.995 | 1.03 |
| 17-330 | AC | 3X | 1 | 0 | 0 | 0 | 0 | 0 | 0 | 6.844 | 1.285 |
| 17-330 | CT | 0 | 1 | 0 | 0 | 0 | 0 | 0 | 0 | 11.071 | 2.21 |
| SeaStar | FU | 3X | 2 | 60 | 70 | 65 | 90 | 90 | 90 | 1.055 | 0.704 |
| 17-726 | SX | X | 2 | 0 | 0 | 0 | 0 | 0 | 0 | 9.566 | 2.076 |
| Bermuda | CT | 0 | 2 | 0 | 0 | 0 | 0 | 40 | 20 | 6.904 | 2.83 |
| 17-726 | AC | 3X | 2 | 0 | 2 | 1 | 0 | 0 | 0 | 6.393 | 1.318 |
| Platinum | SX | 3X | 2 | 40 | 50 | 45 | 80 | 75 | 77.5 | 1.492 | 0.913 |
| Platinum | CT | 0 | 2 | 0 | 0 | 0 | 0 | 0 | 0 | 9.954 | 2.388 |
| SeaStar | AC | X | 2 | 35 | 35 | 35 | 25 | 60 | 42.5 | 1.202 | 0.566 |
| 17-726 | AC | X | 2 | 0 | 2 | 1 | 0 | 0 | 0 | 11.442 | 2.401 |
| Bermuda | AC | 3X | 2 | 40 | 40 | 40 | 50 | 70 | 60 | 2.071 | 1.072 |
| 17-330 | FU | X | 2 | 0 | 0 | 0 | 0 | 0 | 0 | 4.519 | 0.987 |
| 17-726 | FU | 3X | 2 | 25 | 5 | 15 | 0 | 0 | 0 | 4.72 | 1.033 |
| Platinum | AC | X | 2 | 40 | 35 | 37.5 | 15 | 20 | 17.5 | 4.764 | 1.42 |
| SeaStar | SX | X | 2 | 30 | 25 | 27.5 | 20 | 40 | 30 | 2.268 | 0.709 |
| 17-330 | CT | 0 | 2 | 0 | 0 | 0 | 0 | 5 | 2.5 | 10.497 | 2.449 |
| Platinum | AC | 3X | 2 | 60 | 60 | 60 | 60 | 70 | 65 | 2.921 | 1.124 |
| Platinum | SX | X | 2 | 25 | 15 | 20 | 15 | 30 | 22.5 | 4.49 | 1.658 |
| Bermuda | SX | 3X | 2 | 50 | 40 | 45 | 75 | 80 | 77.5 | 1.399 | 0.864 |
| Bermuda | AC | X | 2 | 25 | 30 | 27.5 | 30 | 60 | 45 | 1.986 | 1.032 |
| SeaStar | FU | X | 2 | 20 | 15 | 17.5 | 20 | 40 | 30 | 2.775 | 0.936 |
| SeaStar | CT | 0 | 2 | 0 | 0 | 0 | 0 | 2 | 1 | 10.556 | 2.206 |
| 17-726 | CT | 0 | 2 | 0 | 0 | 0 | 0 | 0 | 0 | 7.832 | 1.582 |
| 17-330 | AC | 3X | 2 | 0 | 0 | 0 | 0 | 0 | 0 | 8.316 | 1.0621 |
| 17-330 | FU | 3X | 2 | 15 | 15 | 15 | 15 | 50 | 32.5 | 3.905 | 1.145 |
| Bermuda | SX | X | 2 | 30 | 40 | 35 | 80 | 70 | 75 | 1.765 | 1.061 |
| SeaStar | AC | 3X | 2 | 75 | 65 | 70 | 85 | 80 | 82.5 | 2.009 | 0.919 |
| 17-726 | FU | X | 2 | 0 | 5 | 2.5 | 0 | 0 | 0 | 5.167 | 1.221 |
| Platinum | FU | X | 2 | 15 | 20 | 17.5 | 25 | 25 | 25 | 4.303 | 1.586 |
| 17-330 | SX | 3X | 2 | 0 | 0 | 0 | 0 | 0 | 0 | 12.987 | 2.721 |
| 17-330 | AC | X | 2 | 2 | 0 | 1 | 0 | 0 | 0 | 9.281 | 1.97 |
| Platinum | FU | 3X | 2 | 40 | 40 | 40 | 60 | 60 | 60 | 3.97 | 1.82 |
| Bermuda | FU | X | 2 | 10 | 25 | 17.5 | 25 | 40 | 32.5 | 2.555 | 1.384 |
| SeaStar | SX | 3X | 2 | 90 | 80 | 85 | 95 | 95 | 95 | 0.726 | 0.522 |
| Bermuda | FU | 3X | 2 | 60 | 40 | 50 | 90 | 75 | 82.5 | 1.201 | 0.749 |
| 17-330 | SX | X | 2 | 5 | 2 | 3.5 | 0 | 5 | 2.5 | 4.721 | 1.03 |
| 17-726 | SX | 3X | 2 | 20 | 20 | 20 | 10 | 10 | 10 | 4.758 | 1.34 |
| 17-726 | AC | 3X | 3 | 0 | 0 | 0 | 0 | 0 | 0 | 7.412 | 1.536 |
| Platinum | SX | X | 3 | 20 | 30 | 25 | 25 | 40 | 32.5 | 6.34 | 2.322 |
| Platinum | AC | X | 3 | 30 | 50 | 40 | 30 | 50 | 40 | 5.155 | 1.634 |
| Bermuda | CT | 0 | 3 | 0 | 0 | 0 | 0 | 0 | 0 | 7.907 | 2.511 |
| Platinum | SX | 3X | 3 | 75 | 70 | 72.5 | 60 | 80 | 70 | 1.916 | 1.268 |
| SeaStar | AC | 3X | 3 | 95 | 85 | 90 | 95 | 95 | 95 | 1.113 | 0.819 |
| 17-726 | AC | X | 3 | 0 | 0 | 0 | 0 | 0 | 0 | 5.721 | 1.135 |
| Bermuda | SX | X | 3 | 20 | 25 | 22.5 | 25 | 40 | 32.5 | 2.119 | 1.213 |
| 17-330 | FU | 3X | 3 | 0 | 0 | 0 | 0 | 0 | 0 | 4.369 | 1.127 |
| Bermuda | FU | X | 3 | 15 | 30 | 22.5 | 40 | 80 | 60 | 1.605 | 1.001 |
| Bermuda | SX | 3X | 3 | 30 | 40 | 35 | 60 | 80 | 70 | 1.552 | 0.995 |
| 17-726 | FU | 3X | 3 | 0 | 0 | 0 | 0 | 0 | 0 | 5.634 | 1.218 |
| Platinum | FU | 3X | 3 | 25 | 30 | 27.5 | 60 | 50 | 55 | 1.86 | 0.935 |
| Platinum | CT | 0 | 3 | 0 | 0 | 0 | 0 | 0 | 0 | 11.239 | 2.344 |
| Platinum | FU | X | 3 | 5 | 10 | 7.5 | 15 | 20 | 17.5 | 3.018 | 1.043 |
| Bermuda | FU | 3X | 3 | 10 | 25 | 17.5 | 50 | 40 | 45 | 2.805 | 1.601 |
| SeaStar | CT | 0 | 3 | 0 | 0 | 0 | 0 | 0 | 0 | 10.825 | 2.206 |
| 17-330 | SX | X | 3 | 0 | 0 | 0 | 0 | 0 | 0 | 8.678 | 1.679 |
| SeaStar | FU | X | 3 | 20 | 15 | 17.5 | 15 | 40 | 27.5 | 3.495 | 1.08 |
| 17-726 | SX | X | 3 | 0 | 0 | 0 | 0 | 0 | 0 | 7.073 | 1.444 |
| SeaStar | SX | X | 3 | 30 | 20 | 25 | 30 | 50 | 40 | 1.896 | 0.65 |
| SeaStar | SX | 3X | 3 | 95 | 75 | 85 | 95 | 95 | 95 | 0.797 | 0.556 |
| Bermuda | AC | 3X | 3 | 5 | 25 | 15 | 80 | 80 | 80 | 8.116 | 0.46 |
| 17-330 | AC | 3X | 3 | 0 | 0 | 0 | 0 | 0 | 0 | 7.218 | 1.545 |
| SeaStar | AC | X | 3 | 80 | 60 | 70 | 90 | 90 | 90 | 0.964 | 0.628 |
| 17-330 | CT | 0 | 3 | 0 | 0 | 0 | 0 | 0 | 0 | 10.356 | 2.109 |
| 17-330 | FU | X | 3 | 0 | 0 | 0 | 0 | 0 | 0 | 8.158 | 1.779 |
| 17-330 | AC | X | 3 | 0 | 0 | 0 | 0 | 0 | 0 | 9.194 | 1.93 |
| 17-330 | SX | 3X | 3 | 0 | 0 | 0 | 5 | 10 | 7.5 | 3.788 | 1.047 |
| 17-726 | FU | X | 3 | 0 | 0 | 0 | 0 | 0 | 0 | 12.46 | 2.713 |
| Bermuda | AC | X | 3 | 10 | 35 | 22.5 | 20 | 40 | 30 | 3.156 | 1.481 |
| Platinum | AC | 3X | 3 | 60 | 50 | 55 | 30 | 50 | 40 | 2.269 | 1.105 |
| SeaStar | FU | 3X | 3 | 80 | 60 | 70 | 75 | 80 | 77.5 | 1.551 | 0.719 |
| 17-726 | CT | 0 | 3 | 0 | 0 | 0 | 0 | 0 | 0 | 9.159 | 1.874 |
| 17-726 | SX | 3X | 3 | 5 | 0 | 2.5 | 0 | 0 | 0 | 8.441 | 1.568 |
| Bermuda | FU | 3X | 4 | 15 | 40 | 27.5 | 60 | 75 | 67.5 | 1.303 | 0.877 |
| Bermuda | CT | 0 | 4 | 0 | 0 | 0 | 0 | 0 | 0 | 8.087 | 2.451 |
| Bermuda | SX | X | 4 | 25 | 40 | 32.5 | 75 | 60 | 67.5 | 1.755 | 1.013 |
| 17-330 | SX | 3X | 4 | 10 | 0 | 5 | 0 | 0 | 0 | 8.662 | 1.563 |
| SeaStar | SX | X | 4 | 25 | 25 | 25 | 30 | 50 | 40 | 1.344 | 1.061 |
| 17-330 | CT | 0 | 4 | 5 | 5 | 5 | 5 | 10 | 7.5 | 5.854 | 1.374 |
| Platinum | SX | 3X | 4 | 75 | 60 | 67.5 | 60 | 70 | 65 | 1.662 | 0.983 |
| 17-330 | SX | X | 4 | 0 | 0 | 0 | 0 | 0 | 0 | 9.261 | 1.883 |
| Bermuda | FU | X | 4 | 25 | 35 | 30 | 50 | 50 | 50 | 1.999 | 1.084 |
| 17-330 | AC | X | 4 | 5 | 5 | 5 | 5 | 10 | 7.5 | 3.027 | 0.822 |
| 17-726 | FU | 3X | 4 | 0 | 0 | 0 | 0 | 0 | 0 | 7.217 | 1.757 |
| Platinum | SX | X | 4 | 25 | 25 | 25 | 10 | 40 | 25 | 4.197 | 1.485 |
| Platinum | AC | 3X | 4 | 75 | 50 | 62.5 | 40 | 60 | 50 | 3.534 | 1.625 |
| SeaStar | SX | 3X | 4 | 90 | 55 | 72.5 | 75 | 70 | 72.5 | 1.624 | 0.851 |
| SeaStar | FU | X | 4 | 20 | 15 | 17.5 | 30 | 40 | 35 | 2.69 | 1.086 |
| 17-330 | FU | X | 4 | 0 | 0 | 0 | 0 | 0 | 0 | 6.545 | 1.371 |
| 17-726 | SX | 3X | 4 | 5 | 0 | 2.5 | 5 | 5 | 5 | 9.667 | 2.213 |
| Platinum | FU | X | 4 | 15 | 25 | 20 | 10 | 25 | 17.5 | 3.622 | 1.239 |
| 17-726 | SX | X | 4 | 0 | 0 | 0 | 0 | 0 | 0 | 10.736 | 2.396 |
| Platinum | FU | 3X | 4 | 30 | 50 | 40 | 60 | 75 | 67.5 | 2.047 | 1.144 |
| 17-330 | FU | 3X | 4 | 0 | 0 | 0 | 0 | 5 | 2.5 | 5.559 | 1.421 |
| SeaStar | CT | 0 | 4 | 0 | 0 | 0 | 0 | 0 | 0 | 11.25 | 2.373 |
| Platinum | AC | X | 4 | 20 | 40 | 30 | 10 | 20 | 15 | 2.975 | 0.991 |
| 17-726 | AC | X | 4 | 0 | 0 | 0 | 0 | 0 | 0 | 4.446 | 0.826 |
| 17-726 | FU | X | 4 | 5 | 5 | 5 | 0 | 0 | 0 | 8.626 | 1.754 |
| SeaStar | AC | 3X | 4 | 50 | 30 | 40 | 95 | 85 | 90 | 1.89 | 1.117 |
| Platinum | CT | 0 | 4 | 0 | 0 | 0 | 0 | 0 | 0 | 13.134 | 2.893 |
| Bermuda | SX | 3X | 4 | 25 | 40 | 32.5 | 75 | 70 | 72.5 | 1.408 | 0.793 |
| 17-330 | AC | 3X | 4 | 10 | 10 | 10 | 15 | 10 | 12.5 | 5.038 | 1.308 |
| 17-726 | CT | 0 | 4 | 0 | 0 | 0 | 0 | 0 | 0 | 8.044 | 1.606 |
| SeaStar | AC | X | 4 | 25 | 15 | 20 | 25 | 40 | 32.5 | 4.742 | 1.444 |
| Bermuda | AC | 3X | 4 | 60 | 60 | 60 | 90 | 90 | 90 | 0.889 | 0.522 |
| SeaStar | FU | 3X | 4 | 65 | 60 | 62.5 | 85 | 80 | 82.5 | 1.256 | 0.705 |
| 17-726 | AC | 3X | 4 | 5 | 0 | 2.5 | 0 | 0 | 0 | 7.207 | 1.489 |
| Bermuda | AC | X | 4 | 10 | 30 | 20 | 25 | 35 | 30 | 2.813 | 1.127 |
| Bermuda | FU | 3X | 5 | 50 | 50 | 50 | 75 | 70 | 72.5 | 1.104 | 0.597 |
| 17-726 | FU | X | 5 | 5 | 0 | 2.5 | 0 | 0 | 0 | 10.174 | 2.296 |
| Platinum | FU | 3X | 5 | 30 | 45 | 37.5 | 40 | 60 | 50 | 3.168 | 1.334 |
| 17-330 | FU | X | 5 | 5 | 0 | 2.5 | 0 | 0 | 0 | 4.029 | 0.792 |
| SeaStar | CT | 0 | 5 | 0 | 0 | 0 | 0 | 5 | 2.5 | 8.928 | 2.002 |
| Bermuda | AC | X | 5 | 60 | 30 | 45 | 40 | 40 | 40 | 1.931 | 0.859 |
| SeaStar | SX | 3X | 5 | 75 | 70 | 72.5 | 75 | 75 | 75 | 1.329 | 0.742 |
| Bermuda | SX | X | 5 | 20 | 40 | 30 | 60 | 70 | 65 | 1.481 | 0.78 |
| 17-330 | CT | 0 | 5 | 5 | 0 | 2.5 | 0 | 0 | 0 | 6.982 | 1.34 |
| SeaStar | AC | X | 5 | 60 | 50 | 55 | 75 | 60 | 67.5 | 1.607 | 0.713 |
| 17-330 | AC | X | 5 | 0 | 0 | 0 | 0 | 0 | 0 | 6.804 | 1.363 |
| Bermuda | FU | X | 5 | 30 | 35 | 32.5 | 30 | 40 | 35 | 1.629 | 0.79 |
| 17-726 | CT | 0 | 5 | 0 | 0 | 0 | 0 | 0 | 0 | 10.275 | 1.896 |
| Bermuda | CT | 0 | 5 | 0 | 0 | 0 | 0 | 0 | 0 | 5.619 | 1.944 |
| 17-726 | FU | 3X | 5 | 0 | 5 | 2.5 | 0 | 0 | 0 | 7.61 | 1.489 |
| SeaStar | FU | X | 5 | 25 | 20 | 22.5 | 20 | 40 | 30 | 2.24 | 0.769 |
| Platinum | CT | 0 | 5 | 0 | 0 | 0 | 0 | 0 | 0 | 9.164 | 1.815 |
| Platinum | SX | 3X | 5 | 60 | 50 | 55 | 40 | 70 | 55 | 1.84 | 0.843 |
| Platinum | AC | X | 5 | 60 | 40 | 50 | 20 | 30 | 25 | 3.588 | 1.077 |
| 17-726 | AC | 3X | 5 | 0 | 0 | 0 | 0 | 0 | 0 | 4.672 | 0.952 |
| 17-330 | FU | 3X | 5 | 15 | 10 | 12.5 | 15 | 10 | 12.5 | 1.908 | 0.556 |
| SeaStar | FU | 3X | 5 | 30 | 25 | 27.5 | 25 | 50 | 37.5 | 1.18 | 0.425 |
| 17-726 | AC | X | 5 | 0 | 0 | 0 | 0 | 0 | 0 | 9.657 | 1.998 |
| SeaStar | SX | X | 5 | 25 | 30 | 27.5 | 20 | 40 | 30 | 1.524 | 0.54 |
| SeaStar | AC | 3X | 5 | 90 | 85 | 87.5 | 95 | 95 | 95 | 0.948 | 0.596 |
| 17-330 | AC | 3X | 5 | 0 | 0 | 0 | 0 | 0 | 0 | 5.594 | 1.188 |
| Platinum | FU | X | 5 | 30 | 20 | 25 | 50 | 20 | 35 | 2.501 | 0.945 |
| 17-726 | SX | X | 5 | 0 | 0 | 0 | 0 | 5 | 2.5 | 11.654 | 2.84 |
| Bermuda | AC | 3X | 5 | 20 | 40 | 30 | 50 | 40 | 45 | 1.93 | 0.912 |
| 17-726 | SX | 3X | 5 | 10 | 5 | 7.5 | 0 | 0 | 0 | 5.357 | 1.112 |
| Bermuda | SX | 3X | 5 | 50 | 50 | 50 | 95 | 95 | 95 | 1.227 | 0.78 |
| 17-330 | SX | 3X | 5 | 15 | 10 | 12.5 | 5 | 2 | 3.5 | 5.692 | 1.004 |
| Platinum | SX | X | 5 | 25 | 20 | 22.5 | 30 | 25 | 27.5 | 2.466 | 0.913 |
| Platinum | AC | 3X | 5 | 50 | 50 | 50 | 40 | 60 | 50 | 2.307 | 1.022 |
| 17-330 | SX | X | 5 | 0 | 0 | 0 | 0 | 0 | 0 | 12.526 | 2.485 |
| Bermuda | CT | 0 | 1 | 0 | 0 | 0 | 0 | 0 | 0 | 5.475 | 1.899 |
| Bermuda | AC | 3X | 1 | 25 | 40 | 32.5 | 50 | 40 | 45 | 2.317 | 1.25 |
| Bermuda | SX | 3X | 1 | 30 | 70 | 50 | 95 | 95 | 95 | 1.325 | 0.868 |
| Bermuda | AC | X | 1 | 10 | 50 | 30 | 20 | 50 | 35 | 3.249 | 1.432 |
| Bermuda | SX | X | 1 | 15 | 35 | 25 | 25 | 50 | 37.5 | 2.367 | 1.323 |
| Bermuda | FU | X | 1 | 15 | 25 | 20 | 15 | 40 | 27.5 | 2.124 | 1.15 |
| Bermuda | FU | 3X | 1 | 25 | 35 | 30 | 10 | 50 | 30 | 1.723 | 0.952 |

We claim:

1. A seed of seashore paspalum variety ‘UGA 17-726’, wherein a representative sample of seed of the variety has been deposited under American Type Culture Collection (ATCC) Accession No. ______.

2. A seed mixture, comprising the seed of claim 1.

3. A seashore paspalum plant of seashore paspalum variety ‘UGA 17-726’ grown from a seed of seashore paspalum variety ‘UGA 17-726’ ATCC Accession No. ______.

4. A plant part of the seashore paspalum plant of claim 3.

5. The seashore paspalum part of claim 4, wherein the plant part is pollen, an ovule or a cell.

6. A tissue culture produced from protoplasts or cells from the seashore paspalum plant of claim 3.

7. The tissue culture of claim 6, wherein the cells or protoplasts are produced from a leaf, stem, protoplast, pollen, ovule, embryo, cotyledon, hypocotyl, meristematic cell, root, root tip, pistil, anther, flower, seed, shoot, stem, pedicel, pod, or petiole.

8. A seashore paspalum plant regenerated from the tissue culture of claim 7.

9. A seashore paspalum plant regenerated from the tissue culture of claim 7, wherein the seashore paspalum plant comprises all of the morphological and physiological properties of a seashore paspalum plant grown from a seed deposited under ATCC Accession No. ______.

10. A method of producing seashore paspalum seed, comprising:
crossing the seashore paspalum plant of claim 3 with itself or a second seashore paspalum plant; and
harvesting a resulting seashore paspalum seed.

11. A seashore paspalum seed produced by the method of claim 10.

12. A seashore paspalum plant, or a part thereof, produced by growing the seed of claim 11.

13. The method of claim 10, wherein the second seashore paspalum plant is transgenic.

14. An F₁ hybrid seed produced by the method of claim 10.

15. A method of introducing a desired trait into seashore paspalum variety ‘UGA 17-726’ comprising:
(a) crossing the plant of claim 3 with a second plant comprising a desired trait to produce F₁ progeny plants;
(b) selecting F₁ progeny plants that have the desired trait to produce selected F₁ progeny plants;
(c) crossing the selected progeny plants with at least a first plant of variety ‘UGA 17-726’ to produce backcross progeny plants;
(d) selecting backcross progeny plants that have the desired trait and physiological and morphological characteristics of seashore paspalum variety ‘UGA 17-726’ to produce selected backcross progeny plants; and
(e) repeating steps (c) and (d) one or more times in succession to produce selected second or higher backcross progeny plants that comprise the desired trait and the physiological and morphological characteristics of seashore paspalum variety ‘UGA 17-726’ when grown in the same environmental conditions.

16. The method of claim 15, wherein the desired trait comprises one or more of herbicide tolerance, resistance to an insect, resistance to a bacterial disease, resistance to a viral disease, resistance to a fungal disease, resistance to a nematode, resistance to a pest, male sterility, site-specific recombination; abiotic stress tolerance, modified phosphorus content, modified antioxidant content, and modified seed yield.

17. The plant of claim 3, further comprising a single locus conversion.

18. The plant of claim 17, wherein the single locus conversion is introduced into the plant by backcrossing or genetic transformation.

19. A method of producing a hybrid seashore paspalum plant derived from seashore-paspalum variety ‘UGA 17-726’, comprising:
(a) preparing a progeny plant derived from seashore paspalum variety ‘UGA 17-726’ ATCC Accession No. ______ by crossing the plant of claim 3 with a seashore paspalum plant of a second variety;
(b) crossing the progeny plant with itself or a second plant to produce a seed of a progeny plant of a subsequent generation;
(c) growing a progeny plant of a subsequent generation from said seed and crossing the progeny plant of a subsequent generation with itself or a second plant; and
(d) repeating steps (b) and (c) for an additional 3-10 generations with sufficient inbreeding to produce a hybrid seashore paspalum plant derived from the seashore paspalum variety ‘UGA 17-726’.

20. A plant produced by the method of claim 19.

21. A method of producing a commodity plant product comprising:
obtaining the seashore paspalum plant of claim 3 or a part thereof; and producing the commodity plant product therefrom.

22. The method of claim 21, wherein the commodity plant product is turfgrass, sod, or biomass feedstock.
